(19) United States
(12) Patent Application Publication
(10) Pub. No.: US 2007/0099841 A1
(43) Pub. Date: May 3, 2007

(54) Prodrugs of T3 and T4 with enhanced bioavailability

(75) Inventors: James Scott Moncrief, Christiansburg, VA (US); Travis Mickle, Coralville, IA (US); Lawrence Olon, Bristol, TN (US); Thomas Piccariello, Blacksburg, VA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: New River Pharmaceuticals Inc., Radford, VA (US)

(21) Appl. No.: 11/517,582

(22) Filed: Sep. 8, 2006

Related U.S. Application Data
(60) Provisional application No. 60/714,859, filed on Sep. 8, 2005.
(60) Provisional application No. 60/786,695, filed on Mar. 29, 2006.

(51) Int. Cl. A61K 38/08; A61K 38/06

(52) U.S. Cl. 514/17; 514/18; 530/330; 530/331

(57) ABSTRACT

The invention relates to compositions of amino acid and peptide conjugates comprising T3 and/or T4. The T3 or T4 is covalently attached to at least one amino acid via the N-terminus, the C-terminus, a side chain of the peptide carrier, and/or interspersed within the peptide chain. Also discussed are methods for protecting and administering active agents and methods for treating thyroid disorders.

### CROSS REFERENCE RELATED APPLICATIONS

This application claims benefit under 35 U.S.C. 119(e) to U.S. Provisional application No. 60/714,859 filed Sep. 8, 2005 and U.S. Provisional application No. 60/786,695 filed Mar. 29, 2006; this application claims benefit under 35 U.S.C. 120 to U.S. application Ser. No. 10/136,433 filed on May 2, 2002, and PCT application PCT/US number unknown, filed Sep. 8, 2006, of the same title as set forth above, each of which is hereby incorporated by reference in their entirety.

### FIELD OF THE INVENTION

The present invention relates to pharmaceutical compounds, compositions, and methods of using the same comprising a chemical moiety attached to T3 and T4. These inventions provide a variety of beneficial effects including providing fast or slow release and reducing side effects associated with taking iodothyronine compounds and compositions. The invention also relates to methods for protecting and administering T3 and/or T4 and for treating thyroid disorders. This invention also relates to prodrugs of T3 and T4 that improve the amount of T3 and/or T4 available in the body and at the same time avoid toxic levels from being released. Further, the invention relates to compositions of T3 prodrugs, T4 prodrugs and various combinations thereof, such as a T3 prodrug with T4, a T3 prodrug with T3, a T3 prodrug with a T4 prodrug and T4 and T4 prodrugs with T3, T4 prodrugs with T4.

### BACKGROUND OF THE INVENTION

The thyroid hormones, thyroxine (T4) and triiodothyronine (T3), are tyrosine-based hormones produced by the thyroid gland play a crucial role in metabolic homeostasis and affect the function of virtually every organ system. Thyroxine (T4) and triiodothyronine (T3) act on the body to increase the basal metabolic rate, affect protein synthesis and increase the body's sensitivity to catecholamines (such as adrenaline). An important component in the synthesis is iodine

In healthy individuals, serum concentrations of the thyroid hormones are controlled by a classic negative-feedback system involving the thyroid gland, the pituitary gland, the hypothalamus and peripheral tissues, such as the liver. Thyroid disorders may result not only from defects in the thyroid gland itself, but also from abnormalities of the pituitary or hypothalamus. In response to the thyroid-stimulating hormone (TSH; also known as thyrotropin) produced by the pituitary, the thyroid gland normally releases an estimated 70 to 90 mg of T4 and 15 to 30 mg of T3 into the blood stream per day. Although the healthy thyroid secretes T3, the major portion of T3 in circulation is thought to result from deiodination of T4 by peripheral tissues, particularly the liver. Synthesis and release of TSH by the pituitary is stimulated by thyroid-releasing hormone (TRH) a tripeptide produced by the hypothalamus in response to changes in metabolism caused by low levels of the thyroid hormones.

Thyroid disorders are common and include hyper- and hypothyroidism. Hypothyroidism is typically characterized by an elevated level of TSH, but varies widely in its clinical presentation. Furthermore, while some patients present with obvious clinical symptoms, others require the use of biochemical tests to determine the status of thyroid function. As a result, hypothyroidism is generally considered under diagnosed. In recent years, a number of hypothyroid syndromes with subtle presentations have been identified. Subclinical hypothyroidism refers to a condition marked by normal levels of T4 and T3 with elevated TSH. “Euthyroid sick syndrome” and “low T3 syndrome” refer to a condition where low serum levels of T3 are present but normal TSH and T4 levels are observed. These conditions have been associated with a number of nonthyroidal illnesses including congestive heart failure, clinical depression, mood disorders.

Hypothyroidism is the most common disorder of the thyroid and is manifested through the thyroid gland's inability to produce sufficient thyroid hormone, primarily triiodothyronine (also known as T3). Symptoms associated with hypothyroidism include cold intolerance, lethargy, fatigue, chronic constipation and a variety of hair and skin changes. Although none of these conditions are life threatening, the disease, left untreated, could result in myxedema, coma, or death. The prevalence of overt and subclinical hypothyroidism in adults ranges from 1 to 10%.

T3 is metabolically active via binding nuclear thyroid hormone receptors and modulating transcription of specific genes. T4 is far less active in the regulation of transcription and is generally considered a prohormone. The metabolic effects of T4 result from the conversion of T4 to T3 by deiodinase enzymes in peripheral tissues, and at the subcellular level once T4 enters a target cell. As noted previously, the T3 in circulation is largely the result of T4 to T3 conversion in the liver.

The early symptoms of hypothyroidism include weakness, fatigue, cold intolerance, constipation, weight gain (unintentional), depression, joint or muscle pain, thin and brittle fingernails, thin and brittle hair, paleness. The late symptoms of hypothyroidism include slow speech, dry flaky skin, thickening of the skin, puffy face, hands and feet, decreased taste and smell, thinning of the eyebrows, hoarseness, abnormal menstrual periods.

Additional symptoms of hypothyroidism may include overall swelling, muscle spasms (cramps), muscle pain, muscle atrophy, uncoordinated movement, absent menstruation (Amenorrhea, Lack of Menses), joint stiffness, dry hair, hair loss, facial swelling, drowsiness, appetite loss, ankle, feet, and leg swelling, short stature, separated sutures, and delayed formation or absence of teeth.

Hypothyroidism is usually diagnosed by means of a physical examination which reveals delayed relaxation of muscles during tests of reflexes; Pale, yellow skin; loss of the outer edge of the eyebrows; thin and brittle hair; coarse facial features; brittle nails; firm swelling of the arms and legs; and mental slowing may be noted. Vital signs may reveal slow heart rate, low blood pressure, and low temperature. A chest X-ray may reveal an enlarged heart.

Laboratory tests to determine thyroid function include: T4 test (low), serum TSH (high in primary hypothyroidism, low or low-normal in secondary hypothyroidism). Additional laboratory abnormalities may include determine thyroid function: increased cholesterol levels, increased liver enzymes, increased serum prolactin, low serum sodium, and/or a complete blood count (CBC) that shows anemia.

The overall goal of hypothyroidism treatment is to replace the deficient thyroid hormone. Levothyroxine is the most commonly used medication. The lowest dose effective in relieving symptoms and normalizing the TSH is used. Life-long therapy is needed. Medication must be continued even when symptoms subside. Thyroid hormone levels should be monitored yearly after a stable dose of medication is determined. Life-long medication is usually needed.

The use of the active hormone, T3, as replacement therapy in hypothyroid conditions has met with limited success primarily because occasionally rapid increases in serum concentrations, or “spiking” levels, of this hormone in the serum occur, which could prove dangerous to patients whose cardiac status is compromised. For this reason, therapy with the prohormone, T4, has become the treatment of choice in hypothyroidism since, to be active, it first must be converted to T3, in vivo, a process which eliminates the potential for spiking T3 serum levels and any serious sequela. However, recent studies of T4 suggest that a general decline in a patient's ability to convert T4 to T3 is associated with aging, and also has been observed where stress or concurrent disease is present. Additionally, a deficiency in the T4 to T3 conversion capacity of particular organs or organ systems may exist.

Given the problems associated with the use of either T3 or T4 as thyroid hormone replacement as herein identified, there is a need for an efficient, effective, low-cost and readily available mechanism for the delivery of thyroid hormones and derivatives thereof. Further, there is a need for compositions and methods to treat hypothyroid conditions and control the absorption of T3 in vivo.

Further, a common problem associated with taking synthetic thyroid hormones is controlling the amount of thyroid hormones in the body. Liothyronine (T3) can be taken as a single dose or several times each day, however both means can lead to high levels of T3 after the hormone is taken. High amounts of T3 can cause symptoms such as a rapid heartbeat, insomnia and anxiety. Synthetic preparations of sodium salts of T4, levothyroxine sodium, (Synthroid®, Levoxyl®, Levothroid® and others) are available as tablets. Sodium salts of T3, liothyronine sodium, are available as tablets (Cytomel®) and in an injectable form (Triostat®). A 4:1 mixture of levothyroxine sodium and liothyronine sodium is also marketed in tablets as liotrix (Thyrolar®).

And it is important that the level of thyroid hormones remains constant to prevent adverse side effects. One way in which to regulate the percent of T3 and T4 in the body is by attaching amino acids and peptides to liothyronine (T3) or thyroxine (T4) and thereby controlling the amount of T3 and/or T4 released in the body. This occurs because conversion of the amino acid or peptide prodrug to its active form is limited by cleavage of the amide bond thus, decreasing the potential for release of toxic levels of the active drug.

The effective delivery of a T3 and/or T4 is often critically dependent on the delivery system used. The importance of these systems becomes magnified when patient compliance and of iodothyronine stability are taken under consideration. As mentioned above, the blunting of the T3 “spike” through a modulated release formulation would markedly improve the safety of that drug. In general, increasing the stability of iodothyronine, such as prolonging shelf life or survival in the stomach, will assure dosage reproducibility and perhaps even reduce the number of dosages required which could improve patient compliance.

Additional information regarding T3 and T4 compounds and compositions may be found for instance in U.S. application Ser. No. 10/136,433, filed May 2, 2002, which is hereby incorporated by reference in its entirety. Similarly, information discussing the effect T4 and T3 admixtures have on each other is further discussed in U.S. application Ser. No. 10/701,173, which is hereby incorporated by reference.

There remains a need for compositions that effectively deliver triiodothyronine (T3) and thyroxine (T4). There also remains a need for methods of protecting and controlling the delivery and/or release of triiodothyronine (T3) and/or thyroxine (T4).

Therefore, the need still exists for a drug delivery system, which enables the use of new molecular T3 and T4 compositions that can reduce the technical, regulatory, and financial risks associated with iodothyronine agents while improving their reproducibility, bioavailability, reliability, and sustained release.

The compounds of the invention may be provided in several useful forms. As such, improved methods are needed to make pharmaceutically effective iodothyronine compounds, compositions and methods of using the same with reduced potential for overdose and/or lower side effects.

### BRIEF DESCRIPTION OF THE DRAWINGS

It is to be understood that both the foregoing general description and the following detailed description are exemplary, but are not restrictive, of the invention.

FIG. 1 illustrates a scheme for the attachment of an iodothyronine compound to the N-terminus of a peptide through the iodothyronine's acid functional group;

FIG. 2 illustrates a scheme for the attachment of iodothyronine to the C-terminus of a peptide through the iodothyronine's amine functional group;

FIG. 3 illustrates the synthesis of T3 amino acid and peptide conjugates;

FIG. 4 illustrates the total T3-time concentration curves following oral administration of T3 sodium or G-T3 (12 mg/kg T3 content; HED˜120 mg T3 sodium);

FIG. 5 illustrates the total T3Δ-time concentration curves following oral administration of T3 sodium or G-T3 (12 mg/kg T3 content; HED˜120 mg T3 sodium);

FIG. 6 illustrates individual animal total T3-time concentration curves following oral administration of T3 sodium or G-T3 (12 mg/kg T3 content; HED˜120 mg T3 sodium);

FIG. 7 illustrates individual animal total T3Δ-time concentration curves following oral administration of T3 sodium or G-T3 (12 mg/kg T3 content; HED˜120 mg T3 sodium);

FIG. 8 illustrates TSH-time concentration curves following oral administration of T3 sodium or G-T3 (12 mg/kg T3 content; HED˜120 mg T3 sodium);

FIG. 9 illustrates the total T3-time concentration curves following oral administration of T3 sodium or V-T3 (12 mg/kg T3 content; HED˜120 mg T3 sodium);

FIG. 10 illustrates the total T3Δ-time concentration curves following oral administration of T3 sodium or V-T3 (12 mg/kg T3 content; HED˜120 mg T3 sodium);

FIG. 11 illustrates the total T3-time concentration curves following oral administration of T3 sodium or I-T3 (12 mg/kg T3 content; HED˜120 mg T3 sodium);

FIG. 12 illustrates the total T3Δ-time concentration curves following oral administration of T3 sodium or I-T3 (12 mg/kg T3 content; HED˜120 mg T3 sodium);

FIG. 13 illustrates the total T3-time concentration curves following oral administration of T3 sodium or Y-T3 (12 mg/kg T3 content; HED˜120 mg T3 sodium);

FIG. 14 illustrates the total T3Δ-time concentration curves following oral administration of T3 sodium or Y-T3 (12 mg/kg T3 content; HED˜120 mg T3 sodium);

FIG. 15 illustrates the total T3-time concentration curves following oral administration of T3 sodium or A2-T3 (12 mg/kg T3 content; HED˜120 mg T3 sodium);

FIG. 16 illustrates the total T3Δ-time concentration curves following oral administration of T3 sodium or A2-T3 (12 mg/kg T3 content; HED˜120 mg T3 sodium);

FIG. 17 illustrates the total T3-time concentration curves following oral administration of T3 sodium or P2-T3 (12 mg/kg T3 content; HED˜120 mg T3 sodium);

FIG. 18 illustrates the total T3Δ-time concentration curves following oral administration of T3 sodium or P2-T3 (12 mg/kg T3 content; HED˜120 mg T3 sodium);

FIG. 19 illustrates the total T3-time concentration curves following oral administration of T3 sodium or F2-T3 (12 mg/kg T3 content; HED˜120 mg T3 sodium);

FIG. 20 illustrates the total T3Δ-time concentration curves following oral administration of T3 sodium or F2-T3 (12 mg/kg T3 content; HED˜120 mg T3 sodium);

FIG. 21 illustrates individual total T3-time concentration curves following oral administration of T3 sodium, G-T3, V-T3, I-T3, Y-T3, A2-T3, P2-T3, or F2-T3 (12 mg/kg T3 content; HED˜120 mg T3 sodium);

FIG. 22 illustrates individual total T3Δ-time concentration curves following oral administration of T3 sodium, G-T3, V-T3, I-T3, Y-T3, A2-T3, P2-T3, or F2-T3 (12 mg/kg T3 content; HED˜120 mg T3 sodium);

FIG. 23 illustrates TSH-time concentration curves following oral administration of T3 sodium or V-T3 (12 mg/kg T3 content; HED˜120 mg T3 sodium);

FIG. 24 illustrates TSH-time concentration curves following oral administration of T3 sodium or I-T3 (12 mg/kg T3 content; HED˜120 mg T3 sodium);

FIG. 25 illustrates TSH-time concentration curves following oral administration of T3 sodium or Y-T3 (12 mg/kg T3 content; HED˜120 mg T3 sodium);

FIG. 26 illustrates TSH-time concentration curves following oral administration of T3 sodium or A2-T3 (12 mg/kg T3 content; HED˜120 mg T3 sodium);

FIG. 27 illustrates TSH-time concentration curves following oral administration of T3 sodium or P2-T3 (12 mg/kg T3 content; HED˜120 mg T3 sodium);

FIG. 28 illustrates TSH-time concentration curves following oral administration of T3 sodium or F2-T3 (12 mg/kg T3 content; HED˜120 mg T3 sodium);

FIG. 29 illustrates total TSH-time concentration curves following oral administration of T3 sodium, G-T3, V-T3, I-T3, Y-T3, A2-T3, P2-T3, or F2-T3 (12 mg/kg T3 content; HED˜120 mg T3 sodium).

### DETAILED DESCRIPTION OF THE INVENTION

The invention relates to changing the pharmacokinetic and pharmacological properties of iodothyronine through covalent modification. Covalent attachment of a chemical moiety to iodothyronine may change one or more of the following: the rate of absorption, the extent of absorption, the metabolism, the distribution, and the elimination (ADME pharmacokinertic properties) of iodothyronine. As such, the alteration of one or more of these characteristics may be designed to provide fast or slow release. Additionally, alteration of one or more of these characteristics may reduce the side effects associated with taking iodothyronine

One aspect of the invention includes iodothyronine conjugates that when administered at a normal therapeutic dose the bioavailablility (area under the time-versus-concentration curve; AUC) of iodothyronine provides a pharmaceutically effective amount of iodothyronine. As the dose is increased, however, the bioavailability of the covalently modified iodothyronine relative to the parent iodothyronine begins to decline, particularly for oral dosage forms. At suprapharmacological doses the bioavailability of the iodothyronine conjugate is substantially decreased as compared to the parent iodothyronine. The relative decrease in bioavailability at higher doses decreases or reduces risks associated with doses of the iodothyronine and helps to reduce fluctuation in bioavailability.

The invention provides iodothyronine prodrugs comprising iodothyronine covalently bound to a chemical moiety. The iodothyronine prodrugs can also be characterized as conjugates in that they possess a covalent attachment. They may also be characterized as conditionally bioreversible derivatives (“CBDs”).

In one embodiment, the iodothyronine prodrug (a compound of one of the formulas described herein) may exhibit one or more of the following advantages over free iodothyronines. The iodothyronine prodrug may prevent or reduce side effects. Preferably, the iodothyronine prodrug provides a serum release curve that does not increase above iodothyronine's toxicity level when administered at higher than therapeutic doses. The iodothyronine prodrug may exhibit a reduced rate of iodothyronine absorption and/or an increased rate of clearance compared to the free iodothyronine. The iodothyronine prodrug may also exhibit a steady-state serum release curve. Preferably, the iodothyronine prodrug provides bioavailability but prevents Cₘₐₓ spiking, increased blood serum concentrations, or uneven release profiles associated with current controlled release iodothyronine products. Preferably, the prodrugs are effectively metabolized into individual amino acids by alimentary tract enzymes before reaching systemic circulation.

The invention provides covalent attachment of a triiodothyronine (T3) or thyroxine (T4) to a carrier peptide, also referred to as, peptidic iodothyronine (generally), peptidic triiodothyronine (T3) or peptidic thyroxine (T4) compositions, respectively. The invention covalently attaches the T3 or T4 to a carrier peptide in a peptide-linked manner, to the N-terminus, the C-terminus, or to the amino acid side chain of the carrier peptide. In a more preferred embodiment the attachment is without the use of a linker.

The carrier peptide itself may also serve as an adjuvant. In a preferred embodiment, the T3 or T4 is covalently attached to the N-terminus or the C-terminus of the carrier peptide or amino acid, also referred to as capped T3 and T4 compositions. In another preferred embodiment, the T3 or T4 is covalently attached directly to the amino acid side chain of the carrier peptide or amino acid; also referred to as side chain T3 or T4 compositions.

Iodothyronine may be bound to one or more chemical moieties, denominated X and Z. A chemical moiety can be any moiety that decreases the pharmacological activity of iodothyronine while bound to the chemical moiety as compared to unbound (free) iodothyronine. The attached chemical moiety can be either naturally occurring or synthetic. In one embodiment, the invention provides an iodothyronine prodrug of Formula I:
I—Xₙ-Zₘ (I)
wherein I is an iodothyronine;
each X is independently a chemical moiety;
each Z is independently a chemical moiety that acts as an adjuvant and is different from at least one X;
n is an increment from 1 to 50, preferably 1 to 10; and
m is an increment from 0 to 50, preferably 0.
When m is 0, the iodothyronine prodrug is a compound of Formula (II):
I—Xₙ (II)
wherein each X is independently a chemical moiety.

Formula (II) can also be written to designate the chemical moiety that is physically attached to the iodothyronine:
I—X₁—(X)ₙ₋₁ (III)
wherein I is iodothyronine; X₁ is a chemical moiety, preferably a single amino acid; each X is independently a chemical moiety that is the same as or different from X₁; and n is an increment from 1 to 50.
Compounds, compositions and methods of the invention provide reduced potential for overdose and/or improve iodothyronine's characteristics with regard to high toxicities or suboptimal release profiles.

As used herein, the term iodothyronine compounds refers to a compound of formula IV 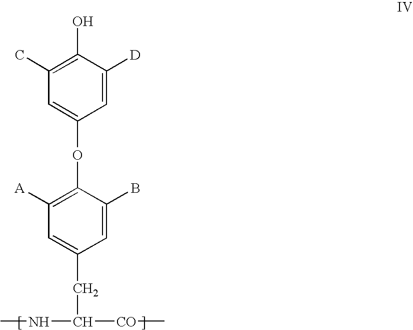
in which A is iodo and B, C and D are independently hydrogen or iodo, each of which are meant to be included as possible compounds which may be utilized as starting compounds on which to base a prodrug of the invention. In particular, thyroxine or T4 is typically referred to as 3:5,3′:5′ tetra-iodothyronine whereas, T3 is typically referred to as 3:5,3′ tri-iodothyronine. In formula IV the NH— is attached to a hydrogen and the CO— of formula IV is attached to a hydroxyl; i.e., NH₂ and COOH, respectively.

A preferred prodrug of the invention is Glycine-3,3′,5-triiodo-L-thyronine hydrochloride. It has a molecular weight of 744.5. Its structure is depicted below. 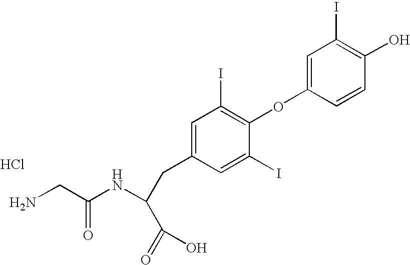
Another preferred prodrug is Glycine-T4 which has a similar structure but includes an additional I.

In the euthyroid or normal state, the thyroid gland secretes both T4 and T3 into the bloodstream; the constant availability of both hormones to target tissues at levels in excess of those possible by peripheral deiodination of T4 alone is essential for optimum health and well being. Thus the invention allows for the mimicry of certain activities of the normal thyroid, namely, the synthesis of a carrier peptide containing the hormones. Following proteolysis of the carrier peptide, the release of the hormones into the bloodstream may be at approximately the same T4:T3 ratio as secreted by the healthy thyroid gland.

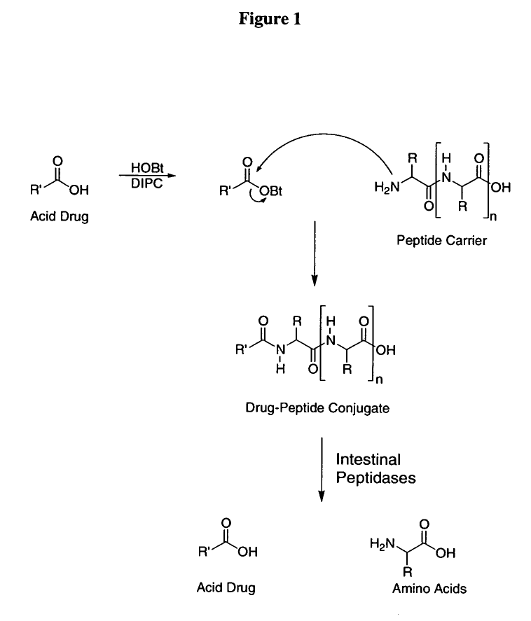

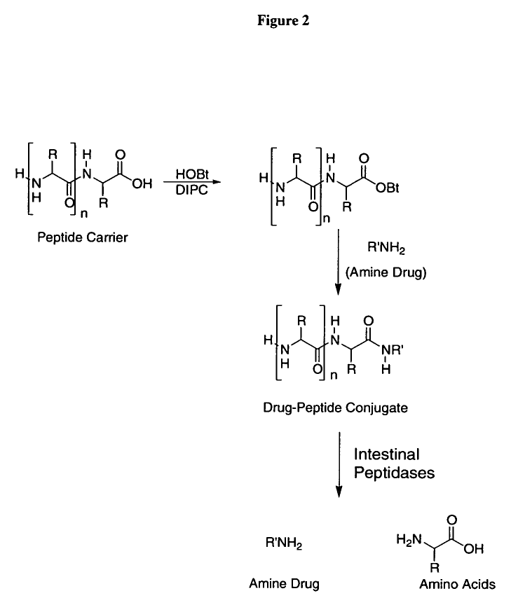

The location of attachment depends on the functional group selected for covalent attachment. For instance, the carboxylic acid of iodothyronine is attached to the N-terminus of the carrier peptide as shown in FIG. 1. Alternatively, the carboxylic acid group can be attached to the side chain of an appropriately substituted amino acid such as lysine. The amine functionality of T3 or T4 is attached to the C-terminus of the carrier peptide as shown in FIG. 2. In both, the C- and N-terminus examples, one monomeric unit forming a new peptide bond in essence, extends the carrier peptide chain. If both the amine and the carboxylic acid of T3 or T4 are used to attach to the carrier peptide, then a peptide-linked interspersed T3 or T4 composition is made. If the alcohol of the T3 or T4 is used to attach to the carrier peptide, then the side chain, the C-terminus or the N-terminus is the point of attachment in order to achieve a stable composition, although a carbonyl, or its equivalent may need to be inserted between the alcohol and the peptide functional group.

In another embodiment of the invention, the iodothyronine-conjugate, i.e., T3-conjugate or T4-conjugate, is covalently bound to: Ala, Arg, Asn, Gln, Gly, His, Ile, Leu, Lys, Met, Phe, Pro, Ser, Thr, Trp, Tyr, Glu, Val, Ala-Ala, Arg-Arg, Asn-Asn, Gln-Gln, Gly-Gly, His-His, Ile-Ile, Leu-Leu, Lys-Lys, Met-Met, Glu-Glu, Phe-Phe, Pro-Pro, Ser-Ser, Thr-Thr, Trp-Trp, Tyr-Tyr, Val-Val, Ala-Ala-Ala, Arg-Arg-Arg, Asn-Asn-Asn, Gln-Gln-Gln, Gly-Gly-Gly, Glu-Glu-Glu, His-His-His, Ile-Ile-Ile, Leu-Leu-Leu, Lys-Lys-Lys, Met-Met-Met, Phe-Phe-Phe, Pro-Pro-Pro, Ser-Ser-Ser, Thr-Thr-Thr, Trp-Trp-Trp, Tyr-Tyr-Tyr or Val-Val-Val. In another preferred embodiment the compound is Gly-T3, Gly-T4, Gly-T3, Ile-T3, Tyr-T3, Ala-Ala-T3, Val-T3, Pro-Pro-T3, Phe-Phe-T3, Glu-Glu-T3, Gly-T4, Ile-T4, Tyr-T4, Ala-Ala-T4, Val-T4, Pro-Pro-T4, Phe-Phe-T4, T4-Glu, T4-Glu-Glu, T4-Lys, Glu-T4, Glu-Glu-T4, or Lys-T4. It should be recognized that the orientation for each of the recited embodiments may be either C-terminus, N-terminus, or side-chained where the amino acid provides for side chain attachment. It should be understood however, that the bound form is directed to covalent bonding and that salt forms are meant to be included. Additionally, these compounds may be in their salt forms for ease of storage or use in formulations.

The invention provides a method for delivering T3 or T4 to a patient, the patient being a human or a non-human animal, comprising administering to the patient compositions of the invention.

The peptidic iodothyronine compositions of the invention have advantages over T4 and T3 alone because the iodothyronine prodrug (conjugate) compositions are a functional surrogate of the naturally occurring thyroglobulin. The methods, compounds and compositions of the invention provide many important advantages and advances. Compositions of the invention may be synthetically produced to alleviate the purity and potency concerns associated other T3 or T4 treatments, compounds and compositions. The methods and compositions of the invention prevent and/or avoid overdosing (e.g., “spiking”). By assuring dosage reproducibility and/or reducing dosage availability, the invention provides the added advantage of improving patient compliance. The invention provides time-release properties to the T3 and/or T4. Providing time-release properties also assures dosage reproducibility and/or reduces the number of dosages required.

In a preferred embodiment, the time-release properties provided by the invention are not dependent upon other commonly used delay release or time-release formulations, such as a microencapsulating matrix during manufacturing. This provides a further advantage of reliable dosing and batch-to-batch reproducibility. This embodiment provides a further advantage of time-release properties without heightened dependence on water solubility of the T3 or T4. As such, time-release properties do not require further formulation such as the dissolution process involved in an enterically coated active agent controlled by pH.

Another advantage provided by preferred embodiments of the invention is the control of T3 or T4 delivery system with regard to molecular weight, molecular size, particle size or combinations thereof. The control of these physical characteristics provided by this embodiment enables predictable diffusion rates and pharmacokinetics.

In a preferred embodiment of the invention, one or more iodothyronine-prodrugs are delivered synergistically. In another embodiment, the compositions of the invention protect the T3 and T4 during storage and/or in passage through the stomach. In a more preferred embodiment, the invention provides methods for protecting, controlling delivery, or controlling release of iodothyronine compounds, or combinations thereof.

In a preferred embodiment, the T3-peptide conjugates, T4-peptide conjugates, combinations thereof, are used in combination with non-bound iodothyronine. These combinations may be administered to a patient with a thyroid related condition comprising administering compounds or compositions described herein to a patient in need thereof. In a preferred embodiment, the condition is hypothyroidism or depression. In another preferred embodiment, the thyroid related conditions include euthyroid goiter, euthyroid sick syndrome, hyperthyroidism, hypothyroidism, thyroiditis, and thyroid cancers. The invention may be used to treat, prevent, or in the prophylaxis of hypothyroidism.

The invention provides the amount of biologically available T3 and/or T4 in a regulated manner and therefore, side effects known from taking too high a dose of liothyronine (T3) and/or thyroxine (T4) can be prevented. The amount of free T3 or free T4 is regulated by the mechanism that cleaves the amide bond and releases the active drug, thereby minimizing the potential for adverse side effects from high doses. In addition, the absorption of the T3 or T4 may be improved.

The invention provides several benefits for T3 and T4 administration, such as but not limited to longer shelf life and the prevention of digestion in the stomach; targeted delivery of the T3 and T4 to specifics sites of action, particularly organ specific; prolonged pharmacologic effect through delayed release of T3 and T4; T3 and T4 can be combined together or with adjuvants to produce synergistic effects; enhanced absorption of the T3 or T4 in the intestinal tract; and formulation for targeted delivery for digestion by intestinal enzymes, intracellular enzymes or blood serum enzymes.

In a preferred embodiment, the carboxylic acid group and the amine group of the T3 or T4 participate in covalent attachment to the carrier peptide, thereby, interspersing the active agent within the carrier peptide in a peptide-linked manner. In another preferred embodiment, the carboxylic acid of the T3 or T4 is covalently attached to the N-terminus of the carrier peptide to produce an amide, referred to herein as “N-capped”. In another preferred embodiment, the amine of the T3 or T4 is covalently attached to the C-terminus of the carrier peptide to produce an amide, referred to herein as “C-capped”.

The invention provides a method for preparing a composition comprising a carrier peptide and a T3 or T4 covalently attached to the carrier peptide. For example attaching the T4 to a side chain of an amino acid to form an active agent/amino acid conjugate may be accomplished as illustrated below. 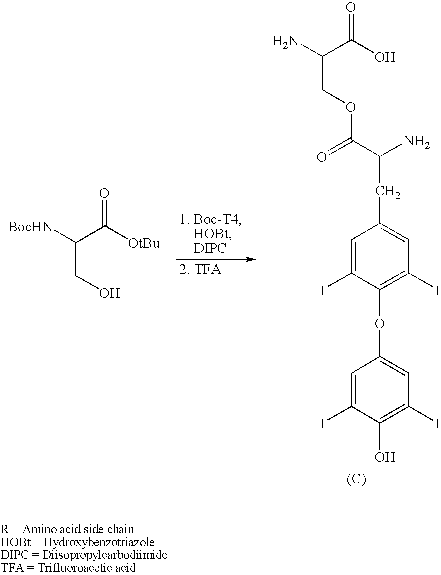
Below is an example of C-terminal attachment of amino acid to T3. 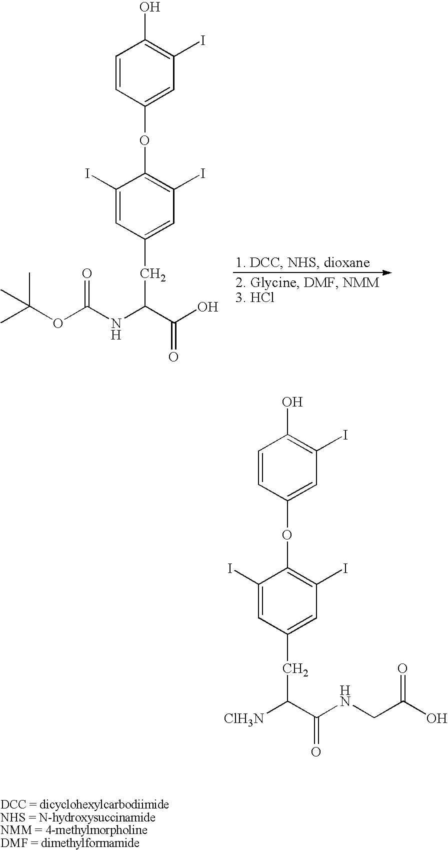
Below is an example of C-terminal attachment of amino acid to T4. 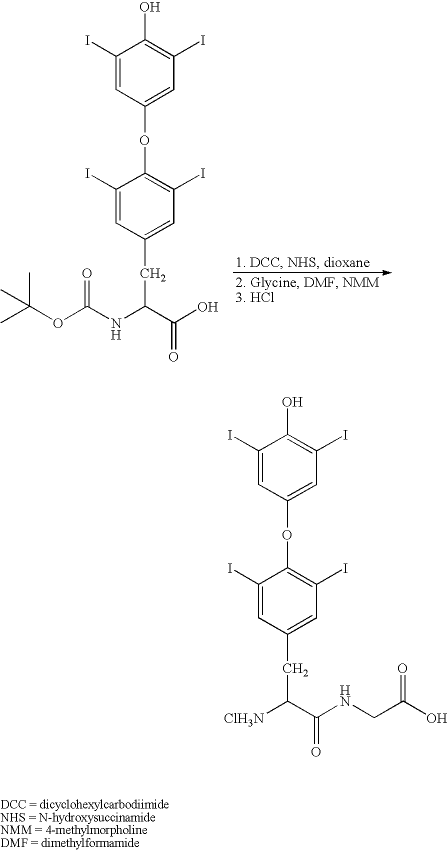
Below is an example of N-terminal attachment of amino acid to T3. 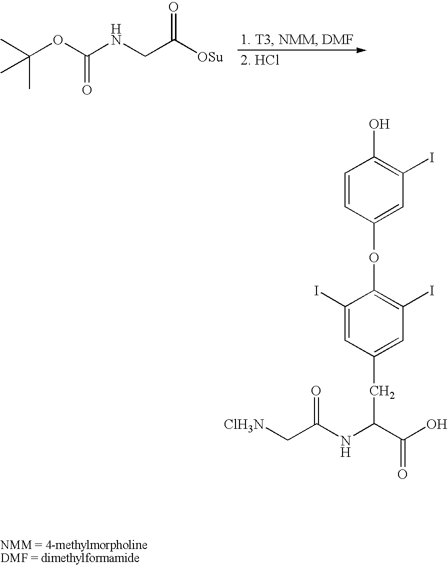
Below is an example of N-terminal attachment of amino acid to T4.

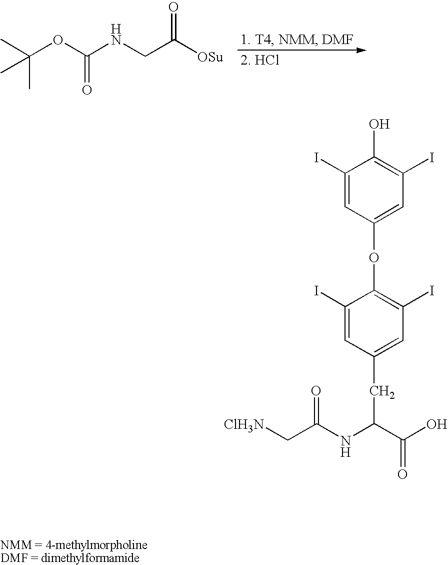

The synthesis of Gly-T4 (HCL salt) is depicted below. 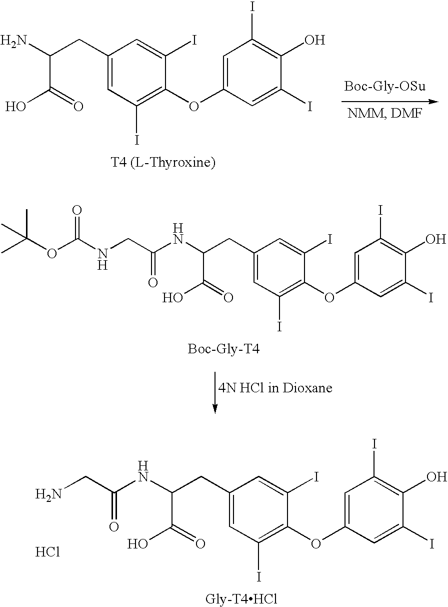
Gly-T4.HCl was characterized by ¹H NMR; purity ˜94%

The synthesis of Gly-T3 (HCl salt) is depicted below. The starting materials in the preparation of NRP409 are 3,3′,5-triiodo-L-thyronine (T3) and Boc-Gly-OSu. 3,3′,5-Triiodo-L-thyronine was obtained from Sigma-Aldrich and Boc-Gly-OSu from BaChem.

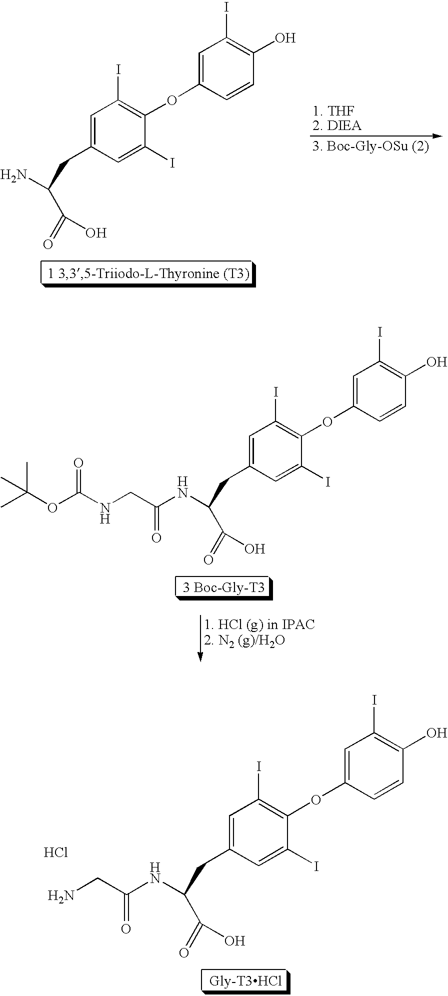

The carrier peptide can be prepared using conventional techniques. If a specific sequence is desired, an automated peptide synthesizer can be used.

Compositions of the invention may comprise the formation of amides from acids and amines and can be prepared by the examples herein. Throughout the application the figures are meant to describe the general scheme of attaching active agents through different functional groups to a variety of peptide conjugates resulting in different embodiments of the invention. One skilled in the art would recognize other reagents, conditions, and properties necessary to conjugate other active agents to other polypeptides from the schemes that are meant to be non-limiting examples. The figures further represent the different embodiments of the invention with regard to length of the active agent conjugate.

The invention teaches broadly a T3-prodrugs and/or T4-prodrugs in combination with unbound T3 and or T4 unbound to form compositions and methods of inventions e.g., T3-prodrugs and unbound T4; T4 prodrug and unbound T3; T3 prodrug, T4 prodrug and unbound T4, etc.

The present T3 and/T4 conjugates may be administering in conjunction with known thyroid drugs such as, but not limited to Synthroid®, Levothyroxine/L-thyroxine, Liothyronine, Liotrix, Methimazole, Propylthiouracil/PTU, Natural thyroid, Thyrotropin alfa, and Time-released T3, compounded.

These products will be used at levels similar to those used in treating hypothyroid patients with current treatments, e.g., Synthroid®, Cytomel®, etc. Determining the precise levels to be used in a particular patient may be accomplished using methods well known to those of skill in the art, including monitoring the levels of thyroid hormones in the blood using known techniques and adjusting the dosage accordingly to get blood levels within acceptable limits. The compositions will be particularly useful in providing oral dosage formulations, for thyroid hormones. While oral dosage formulations are the preferred embodiment for delivery, methods of delivering known iodothyronine compounds may also be utilized.

Iodothyronine may be attached to the carrier peptide through the C-terminus, N-terminus, or side chain of the carrier peptide. Preferably, iodothyronine is attached to the C-terminus of the carrier peptide. It is preferred that aside from attachment of the carrier peptide to the iodothyronine neither is further substituted or protected. In one embodiment, the chemical moiety has one or more free carboxy and/or amine terminal and/or side chain group other than the point of attachment to the iodothyronine. The chemical moiety can be in such a free state, or an ester or salt thereof.

Another embodiment of the invention is a composition or method for safely delivering iodothyronine comprising providing a therapeutically effective amount of iodothyronine which has been covalently bound to a chemical moiety wherein said chemical moiety alters the rate of absorption of the iodothyronine as compared to delivering the unbound iodothyronine. Another embodiment may also provide a means for reducing drug toxicity by altering the rate of clearance of iodothyronine.

Another embodiment of the invention is a composition or method for a sustained-release iodothyronine composition comprising providing iodothyronine which has been covalently bound to a chemical moiety, wherein said chemical moiety provides release of iodothyronine at a rate where the level of iodothyronine is within the therapeutic range but below toxic levels over an extended periods of time, e.g., 8-24 hours or greater.

Another embodiment of the invention is a composition or method for reducing bioavailability or preventing a toxic release profile of iodothyronine comprising iodothyronine covalently bound to a chemical moiety wherein said bound iodothyronine maintains a steady-state serum release curve which provides a therapeutically effective bioavailability but prevents spiking or increase blood serum concentrations compared to unbound iodothyronine.

Another embodiment of the invention is a composition or method for preventing a Cₘₐₓ spike and/or providing a more consistent release curve for iodothyronine while still providing a therapeutically effective bioavailability curve comprising iodothyronine that has been covalently bound to a chemical moiety.

Another embodiment of the invention is a method for reducing or preventing toxicity and/or improving the release and/or providing a steady state of release of a pharmaceutical composition, comprising providing, administering, or prescribing said composition to a human in need thereof, wherein said composition comprises a chemical moiety covalently attached to iodothyronine.

For each of the recited methods of the invention the following properties may be achieved through bonding iodothyronine to the chemical moiety. In one embodiment, the toxicity of the compound may be substantially lower than that of the iodothyronine when delivered in its unbound state or as a salt thereof. In another embodiment, the possibility of overdose/toxicity by oral administration is reduced or eliminated.

The compositions and methods of the invention provide iodothyronine, which when bound to the chemical moiety provide safer and/or more effective dosages for iodothyronine through improved bioavailability curves and/or safer Cₘₐₓ and/or reduce area under the curve for bioavailability.

Preferably, the iodothyronine prodrug exhibits an oral bioavailability of iodothyronine of at least about 60% AUC (area under the curve), more preferably at least about 70%, 80%, 90%, 95%, 96%, 97%, 98%, 99%, compared to unbound iodothyronine.

In one embodiment, the iodothyronine prodrug provides pharmacological parameters (AUC, Cₘₐₓ, Tₘₐₓ, Cₘᵢₙ and/or t_{1/2}) within 80% to 125%, 80% to 120%, 85% to 125%, 90% to 110%, or increments therein of unbound iodothyronine or current commercial product utilized for treatment, e.g., Synthroid®, Cytomel®. It should be recognized that the ranges can, but need not be symmetrical, e.g., 85% to 105%.

In another embodiment, the toxicity of the iodothyronine prodrug is substantially lower than that of the unbound iodothyronine. For example, in a preferred embodiment, the acute toxicity is 1-fold, 2-fold, 3-fold, 4-fold, 5-fold, 6-fold, 7-fold, 8-fold, 9-fold, 10-fold less, or increments therein less lethal than oral administration of unbound iodothyronine.

In accordance with the invention and as used herein, the following terms are defined with the following meanings, unless explicitly stated otherwise.

The compounds, compositions and methods of the invention utilize “iodothyronine conjugates,” which are also referred to as iodothyronine prodrugs.

“Iodothyronine” refers broadly to triiodothyronine (T3), liothyronine, 3,5-diiodothyronine (3,5-T2), 3,3′-diiodothyronine (3,3′-T2), reverse triiodothyronine (3,3′,5′-triiodothyronine, rT3), and 3-monoiodothyronine (3-T1), thyronine (T4), diiodotyrosine, and iodotyrosine.

Throughout this application the use of “chemical moiety”—sometimes referred to as the “conjugate” or the “carrier”—is meant to include any chemical substance, naturally occurring or synthetic that decreases the pharmacological activity until the iodothyronine is released including at least carrier peptides, glycopeptides, carbohydrates, lipids, nucleic acids, nucleosides, or vitamins. Preferably, the chemical moiety is generally recognized as safe (“GRAS”).

Throughout this application the use of “carrier peptide” is meant to include naturally occurring amino acids, synthetic amino acids, and combinations thereof. In particular, carrier peptide is meant to include at least a single amino acid, a dipeptide, a tripeptide, an oligopeptide, a polypeptide, or the nucleic acid-amino acids peptides. The carrier peptide can comprise a homopolymer or heteropolymer of naturally occurring or synthetic amino acids.

The use of the term “straight carrier peptide” is meant to include amino acids that are linked via a —C(O)—NH— linkage, also referred to herein as a “peptide bond,” but may be substituted along the side chains of the carrier peptide. Amino acids that are not joined together via a peptide bond or are not exclusively joined through peptide bonds are not meant to fall within the definition of straight carrier peptide.

The use of the term “unsubstituted carrier peptide” is meant to include amino acids that are linked via a —C(O)—NH— linkage, and are not otherwise substituted along the side chains of the carrier peptide. Amino acids that are not joined together via a peptide bond or are not exclusively joined through peptide bonds are not meant to fall within the definition of unsubstituted carrier peptide.

“Oligopeptide” is meant to include from 2 amino acids to 10 amino acids. “Polypeptides” are meant to include from 2 to 50 amino acids.

“Carbohydrates” includes sugars, starches, cellulose, and related compounds. e.g., (CH₂O)ᵣ, wherein n is an integer larger than 2 or Cₙ(H₂O)ₙ₋₁, with n larger than 5. More specific examples include for instance, fructose, glucose, lactose, maltose, sucrose, glyceraldehyde, dihydroxyacetone, erythrose, ribose, ribulose, xylulose, galactose, mannose, sedoheptulose, neuraminic acid, dextrin, and glycogen.

A “glycoprotein” is a compound containing carbohydrate (or glycan) covalently linked to protein. The carbohydrate may be in the form of a monosaccharide, disaccharide(s), oligosaccharide(s), polysaccharide(s), or their derivatives (e.g. sulfo- or phospho-substituted).

A “glycopeptide” is a compound consisting of carbohydrate linked to an oligopeptide composed of L- and/or D-amino acids. A glyco-amino-acid is a saccharide attached to a single amino acid by any kind of covalent bond. A glycosyl-amino-acid is a compound consisting of saccharide linked through a glycosyl linkage (O—, N— or S—) to an amino acid.

The “carrier range” or “carrier size” is determined based on the effect desired. It is preferably between one to 12 chemical moieties with one to 8 moieties being preferred. In another embodiment the number of chemical moieties attached is a specific number e.g., 1, 2, 3, 4, 5, 6, 7, 8, 9, or 10, etc. Alternatively, the chemical moiety may be described based on its molecular weight. It is preferred that the conjugate weight is below about 2,500 kD, more preferably below about 1,000 kD and most preferably below about 500 kD.

A “composition” as used herein, refers broadly to any composition containing an iodothyronine conjugate. A “pharmaceutical composition” refers to any composition containing an iodothyronine conjugate that only comprises components that are acceptable for pharmaceutical uses, e.g., excludes iodothyronine conjugates for immunological purposes.

Use of phrases such as “decreased”, “reduced”, “diminished”, or “lowered” includes at least a 10% change in pharmacological activity with respect to at least one ADME characteristic or at least one of AUC, Cₘₐₓ, Tₘₐₓ, Cₘᵢₙ, and t_{1/2}. For instance, the change may also be greater than 25%, 35%, 45%, 55%, 65%, 75%, 85%, 95%, 96%, 97%, 98%, 99%, or other increments.

Use of the phrase “similar pharmacological activity” means that two compounds exhibit curves that have substantially the same AUC, Cₘₐₓ, Tₘₐₓ, Cₘᵢₙ, and/or t_{1/2} parameters, preferably within about 30% of each other, more preferably within about 25%, 20%, 10%, 5%, 2%, 1%, or other increments.

“Cₘₐₓ,” is defined as the maximum concentration of free iodothyronine in the body obtained during the dosing interval.

“Tₘₐₓ” is defined as the time to maximum concentration.

“Cₘᵢₙ” is defined as the minimum concentration of iodothyronine in the body after dosing.

“t_{1/2}” is defined as the time required for the amount of iodothyronine in the body to be reduced to one half of its value.

Throughout this application, the term “increment” is used to define a numerical value in varying degrees of precision, e.g., to the nearest 10, 1, 0.1, 0.01, etc. The increment can be rounded to any measurable degree of precision. For example, the range 1 to 100 or increments therein includes ranges such as 20 to 80, 5 to 50, 0.4 to 98, and 0.04 to 98.05.

“Hypothyroidism” as used herein, refers broadly to a condition in which the thyroid gland fails to produce enough thyroid hormone. It is also known as “Myxedema” and “Adult hypothyroidism”

“Thyroid gland” as used herein, refers broadly to the gland located in the front of the neck just below the larynx that secretes hormones which control metabolism. namely, thyroxine (T4) and triiodothyronine (T3).

“Patient” as used herein, refers broadly to any animal that is in need of treatment, most preferably an animal with a thyroid disorder, thyroid condition, or thyroid-related condition. The patient may be a clinical patient such as a human or a veterinary patient such as a companion, domesticated, livestock, exotic, or zoo animal. Animals may be mammals, reptiles, birds, amphibians, or invertebrates.

“Mammal” as used herein, refers broadly to any and all warm-blooded vertebrate animals of the class Mammalia, including humans, non-human primates, felines, canines, rats, pigs, horses, sheep, etc.

“Pretreatment” as used herein, refers broadly to any and all preparation, treatment, or protocol that takes place before receiving an iodothyronine compound or composition of the invention.

“Treating” or “treatment” as used herein, refers broadly to preventing the disease, i.e., causing the clinical symptoms of the disease not to develop in a patient that may be exposed to or predisposed to the disease but does not yet experience or display symptoms of the disease, inhibiting the disease, i.e., arresting or reducing the development of the disease or its clinical symptoms, and/or relieving the disease, i.e., causing regression of the disease or its clinical symptoms. Treatment also encompasses an alleviation of signs and/or symptoms.

“Therapeutically effective amount” as used herein, refers broadly to the amount of a compound that, when administered to a patient for treating a thyroid condition, is sufficient to effect such treatment for a thyroid condition. The “therapeutically effective amount” will vary depending on the compound, the disease and its severity and the age, weight, etc., of the patient to be treated.

“Effective dosage” or “Effective amount” of the iodothyronine prodrug or composition is necessary to treat or provide prophylaxis for a thyroid condition.

“Selection of patients” and “Screening of patients” as used herein, refers broadly to the practice of selecting appropriate patients to receive the treatments described herein. Various factors including but not limited to age, weight, heath history, medications, surgeries, injuries, conditions, illnesses, diseases, infections, gender, ethnicity, genetic markers, polymorphisms, skin color, and sensitivity to T3 or T4 treatment. Still other factors include those used by physicians to determine if a patient is appropriate to receive the treatments described herein.

“Diagnosis” as used herein, refers broadly to the practice of testing, assessing, assaying, and determining whether or not a patient has a thyroid disorder.

“Thyroid disorder” as used herein, refers broadly to include euthyroid goiter, euthyroid sick syndrome, hyperthyroidism, hypothyroidism, depression, thyroiditis, and thyroid cancers, etc.

“Thyroid related condition” as used herein, refers broadly to include euthyroid goiter, euthyroid sick syndrome, hyperthyroidism, hypothyroidism, depression, thyroiditis, and thyroid cancers, etc.

Regarding stereochemistry, this patent is meant to cover all compounds discussed regardless of absolute configurations. Thus, natural, L-amino acids are discussed but the use of D-amino acids are also included.

For each of the embodiments recited herein, the carrier peptide may comprise of one or more of the naturally occurring (L-) amino acids: alanine, arginine, asparagine, aspartic acid, cysteine, glycine, glutamic acid, glutamine, histidine, isoleucine, leucine, lysine, methionine, proline, phenylalanine, serine, tryptophan, threonine, tyrosine, and valine. Another preferred amino acid is beta-alanine. In another embodiment the amino acid or peptide is comprised of one or more of the D-form of the naturally occurring amino acids. In another embodiment the amino acid or peptide is comprised of one or more unnatural, non-standard or synthetic amino acids such as, aminohexanoic acid, biphenylalanine, cyclohexylalanine, cyclohexylglycine, diethylglycine, dipropylglycine, 2,3-diaminoproprionic acid, homophenylalanine, homoserine, homotyrosine, naphthylalanine, norleucine, ornithine, pheylalanine(4-fluoro), phenylalanine(2,3,4,5,6 pentafluoro), phenylalanine(4-nitro), phenylglycine, pipecolic acid, sarcosine, tetrahydroisoquinoline-3-carboxylic acid, and tert-leucine. In another embodiment the amino acid or peptide comprises of one or more amino acid alcohols. In another embodiment the amino acid or peptide comprises of one or more N-methyl amino acids.

In another embodiment, the specific carriers listed in the table may have one or more of amino acids substituted with one of the 20 naturally occurring amino acids. It is preferred that the substitution be with an amino acid which is similar in structure or charge compared to the amino acid in the sequence. For instance, isoleucine (Ile)[I] is structurally very similar to leucine (Leu)[L], whereas, tyrosine (Tyr)[Y] is similar to phenylalanine (Phe)[F], whereas serine (Ser)[S] is similar to threonine (Thr)[T], whereas cysteine (Cys)[C] is similar to methionine (Met)[M], whereas alanine (Ala)[A] is similar to valine (Val)[V], whereas lysine (Lys)[K] is similar to arginine (Arg)[R], whereas asparagine (Asn)[N] is similar to glutamine (Gln)[Q], whereas aspartic acid (Asp)[D] is similar to glutamic acid (Glu)[E], whereas histidine (His)[H] is similar to proline (Pro)[P], and glycine (Gly)[G] is similar to tryptophan (Trp)[W]. In the alternative the preferred amino acid substitutions may be selected according to hydrophilic properties (i.e., polarity) or other common characteristics associated with the 20 essential amino acids. While preferred embodiments utilize the 20 natural amino acids for their GRAS characteristics, it is recognized that minor substitutions along the amino acid chain that do not affect the essential characteristics of the amino are also contemplated.

Herein is a list or where amino acids are grouped according to the characteristics of the side chains:

Aliphatic: Alanine, Glycine, Isoleucine, Leucine, Proline, Valine

Aromatic: Phenylalanine, Tryptophan, Tyrosine

Acidic: Aspartic acid, Glutamic acid

Basic: Arginine, Histidine, Lysine

Hydroxylic: Serine, Threonine

Sulphur-containing: Cysteine, Methionine

Amidic (containing amide group): Asparagine, Glutamine.

The iodothyronine conjugate may also be in salt form. Pharmaceutically acceptable salts, e.g., non-toxic, inorganic and organic acid addition salts, are known in the art. Exemplary salts include, but are not limited to, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, besylate, bicarbonate, bisulfate, bitartrate, borate, butyrate, calcium edetate, camphorate, camphorsulfonate, camsylate, carbonate, citrate, clavulariate, cyclopentanepropionate, digluconate, dodecylsulfate, edetate, edisylate, estolate, esylate, ethanesulfonate, finnarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, isothionate, lactate, lactobionate, laurate, laurylsulphonate, malate, maleate, mandelate, mesylate, methanesulfonate, methylsulfate, mucate, naphthylate, napsylate, nicotinate, nitrate, N-methylglucamine ammonium salt, oleate, oxalate, palmitate, pamoate, pantothenate, pectinate, phosphate, phosphateldiphosphate, picrate, pivalate, polygalacturonate, propionate, p-toluenesulfonate, saccharate, salicylate, stearate, subacetate, succinate, sulfate, sulfosaliculate, suramate, tannate, tartrate, teoclate, thiocyanate, tosylate, triethiodide, undecanoate, and valerate salts, and the like.

In the invention, iodothyronine may be covalently attached to the peptide via a ketone group and a linker. This linker may be a small linear or cyclic molecule containing 2-6 atoms with one or more heteroatoms (such as O, S, N) and one or more functional groups (such as amines, amides, alcohols or acids) or may be made up of a short chain of either amino acids or carbohydrates). For example, glucose would be suitable as a linker.

In yet another embodiment of the invention, linkers can be selected from the group of all chemical classes of compounds such that virtually any side chain of the peptide can be attached. The linker should have a functional pendant group, such as a carboxylate, an alcohol, thiol, oxime, hydraxone, hydrazide, or an amine group, to covalently attach to the carrier peptide. In one preferred embodiment, the alcohol group of iodothyronine is covalently attached to the N-terminus of the peptide via a linker. In another preferred embodiment the ketone group of iodothyronine is attached to a linker through the formation of a ketal and the linker has a pendant group that is attached to the carrier peptide. Examples of linking organic compounds to the N-terminus type of a peptide include, but are not limited to, the attachment of naphthylacetic acid to LH-RH, coumarinic acid to opioid peptides and 1,3-dialkyl-3-acyltriazenes to tetragastrin and pentagastrin. As another example, there are known techniques for forming peptide linked biotin and peptide linked acridine.

In addition to the iodothyronine prodrug, the pharmaceutical compositions of the invention may further comprise one or more pharmaceutical additives. Pharmaceutical additives include a wide range of materials including, but not limited to diluents and bulking substances, binders and adhesives, lubricants, glidants, plasticizers, disintegrants, carrier solvents, buffers, colorants, flavorings, sweeteners, preservatives and stabilizers, adsorbents, and other pharmaceutical additives known in the art.

Lubricants include, but are not limited to, magnesium stearate, calcium stearate, zinc stearate, powdered stearic acid, glyceryl monostearate, glyceryl palmitostearate, glyceryl behenate, silica, magnesium silicate, colloidal silicon dioxide, titanium dioxide, sodium benzoate, sodium lauryl sulfate, sodium stearyl fumarate, hydrogenated vegetable oil, talc, polyethylene glycol, and mineral oil.

Surface agents for formulation include, but are not limited to, sodium lauryl sulfate, dioctyl sodium sulfosuccinate, triethanolamine, polyoxyethylene sorbitan, poloxalkol, and quarternary ammonium salts; excipients such as lactose, mannitol, glucose, fructose, xylose, galactose, sucrose, maltose, xylitol, sorbitol, chloride, sulfate and phosphate salts of potassium, sodium, and magnesium; gelling agents such as colloidal clays; thickening agents such as gum tragacanth or sodium alginate, effervescing mixtures; and wetting agents such as lecithin, polysorbates or laurylsulphates.

Colorants can be used to improve appearance or to help identify the pharmaceutical composition. See 21 C.F.R., Part 74. Exemplary colorants include D&C Red No. 28, D&C Yellow No. 10, FD&C Blue No. 1, FD&C Red No. 40, FD&C Green #3, FD&C Yellow No. 6, and edible inks.

In embodiments where the pharmaceutical composition is compacted into a solid dosage form, e.g., a tablet, a binder can help the ingredients hold together. Binders include, but are not limited to, sugars such as sucrose, lactose, and glucose; corn syrup; soy polysaccharide, gelatin; povidone (e.g., Kollidon®, Plasdone®); Pullulan; cellulose derivatives such as microcrystalline cellulose, hydroxypropylmethyl cellulose (e.g., Methocel®), hydroxypropyl cellulose (e.g., Klucel®), ethylcellulose, hydroxyethyl cellulose, carboxymethylcellulose sodium, and methylcellulose; acrylic and methacrylic acid co-polymers; carbomer (e.g., Carbopol®); polyvinylpolypyrrolidine, polyethylene glycol (Carbowax®); pharmaceutical glaze; alginates such as alginic acid and sodium alginate; gums such as acacia, guar gum, and arabic gums; tragacanth; dextrin and maltodextrin; milk derivatives such as whey; starches such as pregelatinized starch and starch paste; hydrogenated vegetable oil; and magnesium aluminum silicate, as well as other conventional binders known to persons skilled in the art. Exemplary non-limiting bulking substances include sugar, lactose, gelatin, starch, and silicon dioxide.

Glidants can improve the flowability of non-compacted solid dosage forms and can improve the accuracy of dosing. Glidants include, but are not limited to, colloidal silicon dioxide, fumed silicon dioxide, silica gel, talc, magnesium trisilicate, magnesium or calcium stearate, powdered cellulose, starch, and tribasic calcium phosphate.

Plasticizers include, but are not limited to, hydrophobic and/or hydrophilic plasticizers such as, diethyl phthalate, butyl phthalate, diethyl sebacate, dibutyl sebacate, triethyl citrate, acetyltriethyl citrate, acetyltributyl citrate, cronotic acid, propylene glycol, castor oil, triacetin, polyethylene glycol, propylene glycol, glycerin, and sorbitol. Plasticizers are particularly useful for pharmaceutical compositions containing a polymer and in soft capsules and film-coated tablets.

Flavorings improve palatability and may be particularly useful for chewable tablet or liquid dosage forms. Flavorings include, but are not limited to maltol, vanillin, ethyl vanillin, menthol, citric acid, fumaric acid, ethyl maltol, and tartaric acid. Sweeteners include, but are not limited to, sorbitol, saccharin, sodium saccharin, sucrose, aspartame, fructose, mannitol, and invert sugar.

Preservatives and/or stabilizers improving storagability include, but are not limited to, alcohol, sodium benzoate, butylated hydroxy toluene, butylated hydroxyanisole, and ethylenediamine tetraacetic acid.

Disintegrants can increase the dissolution rate of a pharmaceutical composition. Disintegrants include, but are not limited to, alginates such as alginic acid and sodium alginate, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), polyvinylpolypyrrolidine (Plasone-XL®), guar gum, magnesium aluminum silicate, methyl cellulose, microcrystalline cellulose, polacrilin potassium, powdered cellulose, starch, pregelatinized starch, sodium starch glycolate (e.g., Explotab®, Primogel®).

Diluents increase the bulk of a dosage form and may make the dosage form easier to handle. Exemplary diluents include, but are not limited to, lactose, dextrose, saccharose, cellulose, starch, and calcium phosphate for solid dosage forms, e.g., tablets and capsules; olive oil and ethyl oleate for soft capsules; water and vegetable oil for liquid dosage forms, e.g., suspensions and emulsions. Additional suitable diluents include, but are not limited to, sucrose, dextrates, dextrin, maltodextrin, microcrystalline cellulose (e.g., Avicel®), microfine cellulose, powdered cellulose, pregelatinized starch (e.g., Starch 1500®), calcium phosphate dihydrate, soy polysaccharide (e.g., Emcosoy®), gelatin, silicon dioxide, calcium sulfate, calcium carbonate, magnesium carbonate, magnesium oxide, sorbitol, mannitol, kaolin, polymethacrylates (e.g., Eudragit®), potassium chloride, sodium chloride, and talc.

In embodiments where the pharmaceutical composition is formulated for a liquid dosage form, the pharmaceutical composition may include one or more solvents. Suitable solvents include, but are not limited to, water; alcohols such as ethanol and isopropyl alcohol; methylene chloride; vegetable oil; polyethylene glycol; propylene glycol; and glycerin or mixing and combination thereof.

The pharmaceutical composition can comprise a buffer. Buffers include, but are not limited to, lactic acid, citric acid, acetic acid, sodium lactate, sodium citrate, and sodium acetate.

Hydrophilic polymers suitable for use in the sustained release formulation include: one or more natural or partially or totally synthetic hydrophilic gums such as acacia, gum tragacanth, locust bean gum, guar gum, or karaya gum, modified cellulosic substances such as methylcellulose, hydroxomethylcellulose, hydroxypropyl methylcellulose, hydroxypropyl cellulose, hydroxyethylcellulose, carboxymethylcellulose; proteinaceous substances such as agar, pectin, carrageen, and alginates; and other hydrophilic polymers such as carboxypolymethylene, gelatin, casein, zein, bentonite, magnesium aluminum silicate, polysaccharides, modified starch derivatives, and other hydrophilic polymers known to those of skill in the art or a combination of such polymers.

One of ordinary skill in the art would recognize a variety of structures, such as bead constructions and coatings, useful for achieving particular release profiles. It is also possible for the dosage form to combine any forms of release known to persons of ordinary skill in the art. These include immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is known in the art. See, e.g., U.S. Pat. No. 6,913,768.

However, it should be noted that the iodothyronine conjugate controls the release of iodothyronine into the digestive tract over an extended period of time resulting in an improved profile when compared to immediate release combinations and reduces and/or prevents toxicity without the addition of the above additives. In a preferred embodiment no further sustained release additives are required to achieve a blunted or reduced pharmacokinetic curve while achieving therapeutically effective amounts of iodothyronine release.

The dose range for adult human beings will depend on a number of factors including the age, weight and condition of the patient and the administration route. Tablets and other forms of presentation provided in discrete units conveniently contain a daily dose, or an appropriate fraction thereof, of the iodothyronine conjugate. The dosage form can contain a dose of about 2.5 mg to about 500 mg, about 10 mg to about 300 mg, about 10 mg to about 100 mg, about 25 mg to about 75 mg, or increments therein. In a preferred embodiment, the dosage form contains 5 mg, 10 mg, 25 mg, 50 mg, or 100 mg of an iodothyronine prodrug.

Tablets and other dosage forms provided in discrete units can contain a daily dose, or an appropriate fraction thereof, of one or more iodothyronine prodrugs.

Compositions of the invention may be administered in a partial, i.e., fractional dose, one or more times during a 24 hour period, a single dose during a 24 hour period of time, a double dose during a 24 hour period of time, or more than a double dose during a 24 hour period of time. Fractional, double or other multiple doses may be taken simultaneously or at different times during the 24-hour period. The doses may be uneven doses with regard to one another or with regard to the individual components at different administration times. Preferably, a single dose is administered once daily.

Likewise, the compositions of the invention may be provided in a blister pack or other such pharmaceutical package. Further, the compositions of the present inventive subject matter may further include or be accompanied by indicia allowing individuals to identify the compositions as products for a prescribed treatment. The indicia may further additionally include an indication of the above specified time periods for administering the compositions. For example the indicia may be time indicia indicating a specific or general time of day for administration of the composition, or the indicia may be a day indicia indicating a day of the week for administration of the composition. The blister pack or other combination package may also include a second pharmaceutical product.

The compounds of the invention can be administered by a variety of dosage forms. Any biologically acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspension in an aqueous liquid or a non-aqueous liquid, emulsions, tablets, syringes, multi-layer tablets, bi-layer tablets, capsules, soft gelatin capsules, hard gelatin capsules, caplets, lozenges, chewable lozenges, beads, powders, granules, particles, microparticles, dispersible granules, cachets, suppositories, creams, topicals, inhalants, aerosol inhalants, patches, particle inhalants, implants, depot implants, ingestibles, injectables (including subcutaneous, intramuscular, intravenous, and intradermal), infusions, emulsions, health bars, confections, animal feeds, cereals, yogurts, cereal coatings, foods, nutritive foods, functional foods and combinations thereof. Preferably, said composition may be in the form of any of the known varieties of tablets (e.g., chewable tablets, conventional tablets, film-coated tablets, compressed tablets), capsules, liquid dispersions for oral administration (e.g., syrups, emulsions, solutions or suspensions).

However, the most effective means for delivering the abuse-resistant iodothyronine compounds of the invention is orally, to permit maximum release of iodothyronine to provide therapeutic effectiveness and/or sustained release while maintaining abuse resistance. When delivered by the oral route iodothyronine is released into circulation, preferably over an extended period of time as compared to iodothyronine alone.

It is preferred that the iodothyronine conjugate be compact enough to allow for a reduction in overall administration size. The smaller size of the iodothyronine prodrug dosage forms promotes ease of swallowing.

For oral administration, fine powders or granules containing diluting, dispersing and/or surface-active agents may be presented in a draught, in water or a syrup, in capsules or sachets in the dry state, in a non-aqueous suspension wherein suspending agents may be included, or in a suspension in water or a syrup. Where desirable or necessary, flavoring, preserving, suspending, thickening or emulsifying agents can be included.

Preferably, the composition of the invention is in a form suitable for oral administration. Commonly applied oral formulations are further described in US2003/0050344 that is hereby incorporated by reference in its entirety. Additional oral formulations are described in the U.S. Pharmacopeia, Vol. 28, 2005 and can be found at http://www.fda.gov/cder/dsm/DRG/drg00201.htm.

Accordingly, the invention also provides methods comprising providing, administering, prescribing, or consuming an iodothyronine prodrug. The invention also provides pharmaceutical compositions comprising an iodothyronine prodrug. The formulation of such a pharmaceutical composition can optionally enhance or achieve the desired release profile.

Exemplary uses for the prodrugs and compositions are listed below in Table A.

TABLE A
Contemplated Uses of the prodrugs for and combinations thereof

the treatment of depression in substance abusers who are non-responsive to
tricyclic antidepressants
the treatment of Raynaud's disease
the treatment of vasospastic attacks
potentiation of tricyclic antidepressants for the treatment of panic disorders
the treatment of high-grade astrocytomas
in combination with radiation for the treatment of glioblastoma multiforme
the treatment of colonic pseudo-obstruction caused by myxedema
the treatment of septic shock
the treatment of obesity
the reduction of atrial fibrillation following coronary bypass surgery
the reduction in the requirements for cardioversion following coronary
bypass surgery
the reduction in the requirements for anticoagulation following coronary
bypass surgery.
useful as a vasodilator
useful as an inotropic agent
augmentation of selective serotonin reuptake inhibitors to improve
recovery in patients suffering from post-traumatic stress disorder
the treatment of anxious depression
the treatment of chronic schizophrenia
the treatment of Kashin-Beck disease
used to increase the levels of active and/or latent TGF-β
the treatment of dopamine-dependent shock
the treatment of breast cancer
increasing patient responses to refractory depression treated with tricyclic
antidepressant therapy.
increasing cardiac output in patients undergoing coronary bypass surgery
lowering systemic vascular resistance in patients undergoing coronary
bypass surgery
the treatment of psychotic children under the age of 6
the prevention of central nervous system (CNS) ischemia in patients
suffering acute insult
the promotion of scalp hair growth
as a component of a composition useful for immune system stimulation
the amelioration of skin damage resulting from the administration of
irradiation
the induction of an in vivo liver hyperplastic microenvironment that is
conducive to transfection of foreign genes.
as a sensitizing agent to sensitize cancerous cells to cytotoxic agents
coadministration with KGF for the synergistic induction of a semi-
synchronous wave of liver cell proliferation in vivo.
for the directed differentiation of an in vitro culture of stem cells of the
central nervous system of a mammal
the treatment of hyponatremia due to hypothyroidism
to increase lymphocytic responses in patients presented with antigen
to enhance the maturation of kidneys
co-administration with lithium to eliminate rapid-cycling bipolar disorder
for the treatment of von Willebrand syndrome type 1
the treatment of myxedema coma
the treatment of chronic urticaria in patients suffering from thyroid
autoimmunity
the treatment of patients suffering from chronic depression
the treatment of patients suffering from chronic dysthymia
the treatment of patients suffering from dilated cardiomyopathy, by
increasing cardiac output
the treatment of patients suffering from dilated cardiomyopathy, by
increasing heart rate
co-administration with ¹³¹I therapy for the treatment of Graves'
ophthalmopathy
the treatment of patients suffering from severe forms of non-rapid cycling
bipolar affective illness
the treatment of a patient suffering from congenital hypothyroidism
the treatment of a patient suffering from necrotizing enterocolitis
the treatment of benign goiter
the improvement of cardiac transplant survival from hemodynamically
stable donors
administration during anti-thyroid drug treatment for the reduction of
glandular mass
the treatment of sporadic goiter
the treatment of headaches
for the prevention of recurrent nodular goiter
the use of D-thyroxine for the treatment of glycogen storage diseases
type VI and VIa
administration for the reduction of total plasma cholesterol
administration for the reduction of plasma low-density lipoprotein
cholesterol
administration for the reduction of plasma high-density lipoprotein
cholesterol
the treatment of Hashimoto's thyroiditis
administration of D-thyroxine for the treatment of endocrine exophthalmos
the treatment of seasonal affective disorder (SAD)
administration in children for the treatment of cretinism
administration in children to counter the effects of hypothyroidism
for the amelioration of angina pectoris
the treatment of fibrocystic breast disease
the treatment of hyperhomocysteinemia
the treatment of hypertrichosis
the treatment of acanthosis nigricans
the treatment of angioedema
the treatment of nonimmune hydrops fetalis
the treatment of distal renal tubular acidosis
co-administration with a dopamine agonist and a prolactin stimulator to
increase hyperglycemic sensitivity to insulin
co-administration with a dopamine agonist and a prolactin stimulator
to reduce body fat stores
co-administration with a dopamine agonist and a prolactin stimulator
to suppress hyperinsulinemia
co-administration with a dopamine agonist and a prolactin stimulator
to reduce hyperglycemia
to increase intracellular potassium content of lymphocytes of a mammal
co-administration with a tocotrienol for the treatment of diabetes mellitus
co-administration with a tocotrienol for the treatment of fever
co-administration with a tocotrienol for the treatment of pain
co-administration with a tocotrienol for the treatment of chronic fatigue
syndrome
co-administration of with a tocotrienol for the treatment of functio laesa
administration for the restoration of neuronal plasticity (i.e., to treat
degenerative pathologies such as senile dementia like
Alzheimer's disease, Parkinsonism, etc).
as an active component of a hibernation-inducing composition
for the treatment of mastopathy
the treatment of adverse inflammatory effects of certain autoimmune
responses
treatment of coeliac disease in infants
the treatment of subacute (DeQuervain's) thyroiditis
the treatment of multiple-organ dysfunction syndrome
the treatment of cataract conditions, includin cortical cataracts
the treatment of osteoporosis
as useful for accelerating the rate of wound-healing
the treatment of hyperkeratosis

It will be appreciated that the pharmacological activity of the compositions of the invention can be demonstrated using standard pharmacological models that are known in the art. For each of the described embodiments one or more characteristics as described throughout the specification may be realized. It should also be recognized that the compounds and compositions described throughout the specification may be utilized for a variety of novel methods of treatment, reduction of toxicity, improved release profiles, etc. An embodiment may obtain, one or more of: a conjugate with toxicity of iodothyronine that is substantially lower than that of unbound iodothyronine.

### EXAMPLES

Any feature of the above-describe embodiments can be used in combination with any other feature of the above-described embodiments. Synthesis of amino acid and peptide conjugates may be verified using the following analytical methods: Nuclear Magnetic Resonance, High Resolution Mass Spectroscopy or Elemental Analysis and melting point or differential scanning calorimetry (DSC).

In order to facilitate a more complete understanding of the invention, Examples are provided below. However, the scope of the invention is not limited to specific embodiments disclosed in these Examples, which are for purposes of illustration only. For example, while the examples are directed to T3 compounds and compositions it is contemplated that T4 compounds may be prepared and will provided similar characteristics to the T3 compounds and compositions described below.

The following are non-limiting examples preferred carrier peptides that may be made and attached according to the invention: Ala, Arg, Asn, Gln, Gly, His, Ile, Leu, Lys, Met, Phe, Pro, Ser, Thr, Trp, Tyr, Val, Ala₂, Gly₂, Ile₂, Leu₂, Lys₂, Phe₂, Pro₂, Ser₂, Thr₂, Tyr₂, Val₂, Ala₃, Gly₃, Ile₃, Leu₃, Phe₃, Tyr₃, and Val₃.

The following abbreviations are used in the Examples and throughout the patent:
G-T3=Glycine-T3 conjugate
Gly-T3=Glycine-T3 conjugate
Gly-T3=2-(2-aminoacetoamido)-3-(4-(4-hydroxy-3-iodophenoxy)-3,5-diiodophenyl) propanoic acid, hydrochloride salt
N=Number of animals
N/A=Not applicable
NS=No Sample
PO=Oral route
T3=3,3,4-Triiodo-L-thyroxine
TSH=Thyroid stimulating hormone

### Example 1

### Preparation of Amino Acid Succinates

To a solution of the N-protected amino acid (1.0 eq) in dioxane (22 ml/gram of a.a.) was added N-methylmorpholine (1.1 eq) and 1,3-dicyclohexylcarbodiimide (1.1 eq). The solution was allowed to stir overnight at ambient temperature under argon. Then dicyclohexylurea was filtered off and the filtrate concentrated under reduced pressure. The product was recrystalized in acetone/hexane at 0° C. and dried to afford the corresponding N-protected amino acid succinate.

### Example 2

### Preparation of Di- and Tripeptide Succinate

The appropriate amino acid (1.5 eq) was dissolved in N,N-dimethylforamide/dioxane/H₂O (2:2:1). N-Methylmorpholine (3.0 eq) and N-protected amino acid succinate (1.0 eq) were added and the solution was allowed to stir overnight at ambient temperature, under argon. Ethylacetate was then added and the organic layer washed with 2% acetic acid, water, brine and dried over sodium sulfate. The organic extract was concentrated and dried under vacuum to afford the dipeptide. The procedure for synthesis of dipeptide succinates is the same as for synthesis of amino acid succinates. Tripeptide succinates are prepared using the same procedure as for synthesis of dipeptides succinates except that the appropriate N-protected dipeptide succinate is reacted with an amino acid to form the tripeptide and then converted to the succinate.

### Example 3

### Preparation of Protected Amino Acid T3

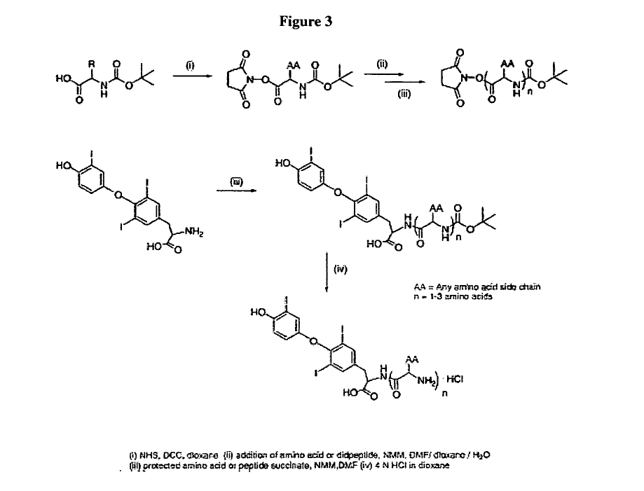

To a mixture of T3 (1.0 eq) in dimethylforamide (10 ml) was added N-methylmorpholine (2.5 eq) and protected amino acid succinate (1.1 eq). The solution was stirred overnight at ambient temperature under argon and then the solvent was removed under reduced pressure. Water was added and the mixture stirred for 15 minutes prior to removing the solvents under reduced pressure. The crude product mixture was dissolved in ethyl acetate, washed with 2½% acetic acid (aq), brine solution and dried over sodium sulfate. The solvent from the organic extract was removed under reduced pressure and purified by preparative HPLC to obtain the desired product. As demonstrated in FIG. 3.

### Example 4

### Deprotection of Protected Amino Acid T3

The protected amino acid amphetamine conjugate was dissolved in a solution of 4 N HCl in dioxane (25 ml) and allowed to stir overnight at ambient temperature under argon. The solvent was then removed under reduced pressure to afford the amino acid conjugate.

### Example 5

### Method of Making Gly-T3 (HCl Salt)

Production of the drug substance Gly-T3.HCl was performed as a one-pot reaction inside a 20-gallon glass lined reactor. To limit exposure of the workers to the potent material, the starting reagent T3 was prepared as a slurry in THF inside an isolator. The resulting slurry was then transferred to the 20-gallon reactor through a Teflon line. Subsequently, Boc-Gly-OSu was dissolved in THF, transferred to the 20-gallon reactor and then DIEA was dissolved in THF, and also transferred to the 20-gallon reactor. The suspension was stirred for 15 hours, at ambient temperature and became a clear solution. In process testing by HPLC (% AUC) of the collected sample after 15 hours showed a purity of 98.5% for the desired compound, Boc-Gly-T3. The reaction was then quenched by adding water and the THF was removed by distillation. The solvent, TBME was then added and the batch was allowed to stir overnight at 5 to 10° C. The solution was acidified by a 20% aqueous Na₂SO₄/NaHSO₄ buffer solution. The product was extracted in TBME and the organic layers were combined in a 60 L Pope™ tank and dried with Na₂SO₄. The dried solution was then filtered through a 0.22 μm filter, charged into the 20-gallon reactor and stirred overnight at 15° C. The TBME was removed by distillation, followed by IPAc chases (continuous feed), while the batch stirred overnight at 15° C. The intermediate was Boc deprotected by pressurizing the 20-gallon reactor headspace with 30 psi HCl gas for 3 hours and 30 minutes. The obtained precipitate was filtered inside the isolator, washed with IPAc, and dried for 110 hours inside a vacuum oven at 35° C. In process testing by GC showed the presence of 6.13% residual IPAc. Therefore, the solids were hydrated (water displacement) for approximately 24 hours and dried until constant weight, in a vacuum oven for 50 hours at 35° C. The material was packaged in glass amber bottles, which were put in polyethylene bags and stored at −20° C. with desiccants.

### Example 6

### Preparation of Dipeptide T3

The procedure for synthesis of a peptide conjugate is the same as for an amino acid conjugate except that the appropriate dipeptide succinate is used instead of the amino acid succinate.

### Example 7

### Preparation of Tripeptide T3

The procedure for synthesis of a tripeptide conjugate is the same as for an amino acid conjugate and then the appropriate dipeptide succinate is reacted with the amino acid conjugate to form the tripeptide.

All reagents were used as received. ¹H NMR was run on a Bruker 300 MHz (300) or JEOL 500 MHz (500) NMR spectrophotometer using tetramethylsilane as an internal standard.

### Example 8

### In Vivo Performance Studies

Materials and Methods of the In Vivo Performance Studies

Solid Dose Oral Delivery

Compounds were tested in Sprague-dawley rats (˜250 g). Defined doses were delivered as capsules containing. Serum was collected from rats at 2, 4, 6, 9, 12 and 24 hours after capsule delivery. Total serum T3 concentrations were determined by ELISA using a commercially available kit (Total Triiodothyronine (Total T3) ELISA KIT, product #1700, ALPHA DIAGNOSTIC, San Antonio, Tex.). Total serum T4 concentrations were determined by ELISA using a commercially available kit (Total Thyronine (Total T4) ELISA KIT, product #1100, ALPHA DIAGNOSTIC, San Antonio, Tex.).

Solution Dose Oral Delivery

Compounds were tested in Sprague-dawley rats (˜250 g). Defined doses were delivered as oral solutions in 0.5% sodium bicarbonate buffer with T3 sodium salt containing 12 mcgT3/kg or triiodothyronine composition containing the equivalent amount of T3. Rats were dosed immediately following 0 hour serum collection. Serum was collected from rats at 2, 4, 6, 9, 12 and 24 hours after capsule delivery. Total serum T3 concentrations were determined by ELISA using a commercially available kit (Total Triiodothyronine (total T3) ELISA KIT, product #1700, ALPHA DIAGNOSTIC, San Antonio, Tex.). Total serum T4 concentrations were determined by ELISA using a commercially available kit (Total Thyronine (Total T4) ELISA KIT, product #1100, ALPHA DIAGNOSTIC, San Antonio, Tex.).

The general procedures described above were employed for the experimental data described below. These procedures are subject to minor variations in timing and weight of rats, etc.

In Vivo Performance Studies Results

AUC and delta-AUC for preferred amino acids and peptide conjugates of T3

| | T3 | G-T3 | V-T3 | I-T3 | Y-T3 | A₂-T3 | P₂-T3 | F₂-T3 |
|---|---|---|---|---|---|---|---|---|
| AUC | 100 | 88 | 95 | 97 | 96 | 104 | 92 | 88 |
| dAUC | 100 | 91 | 99 | 101 | 100 | 113 | 98 | 95 |

Pharmacokinetic Evaluation of Total T3 and TSH Following Oral Administration of T3 Sodium or Amino Acid Conjugate Glycine-T3 HCl (G-T3) in Hypothyroid Rats

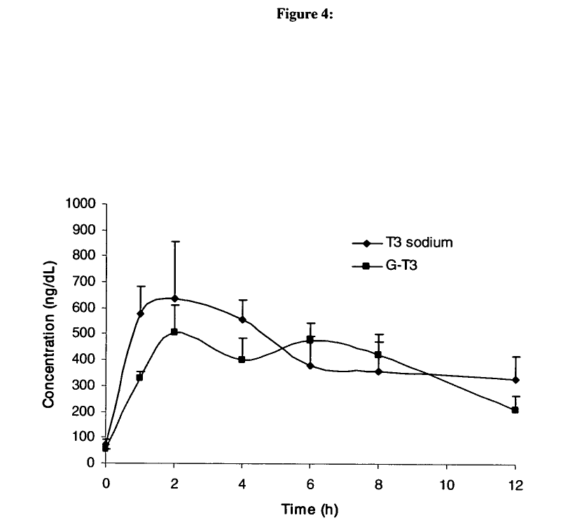

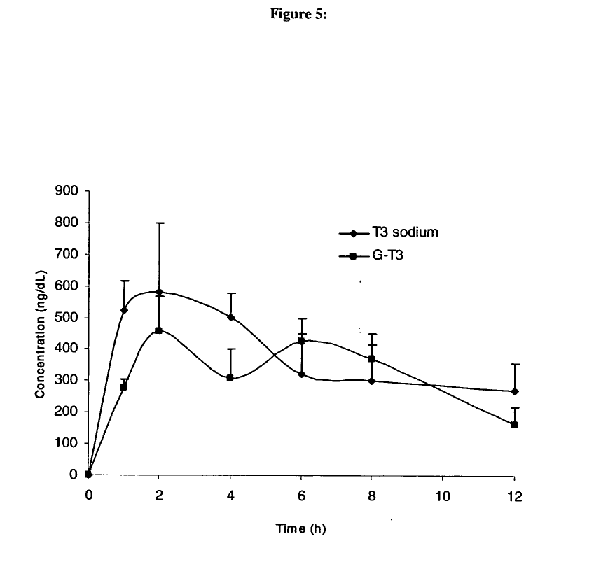

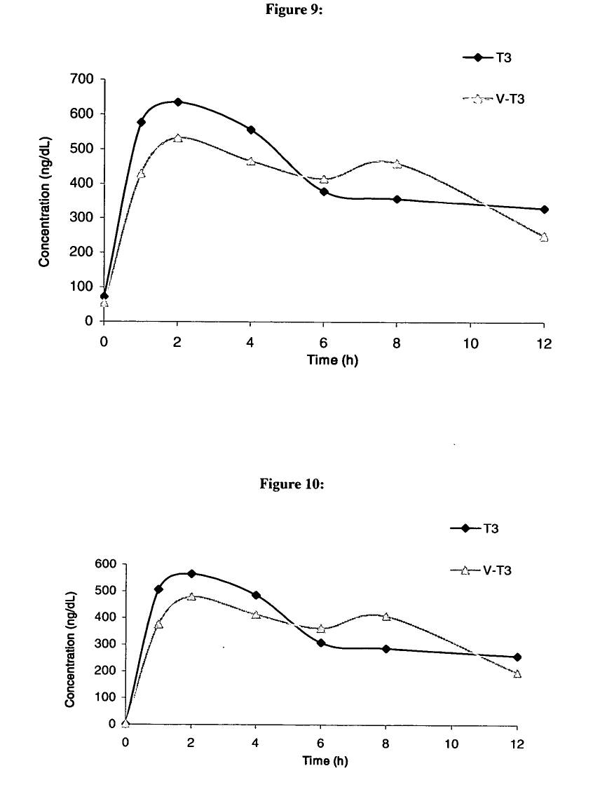

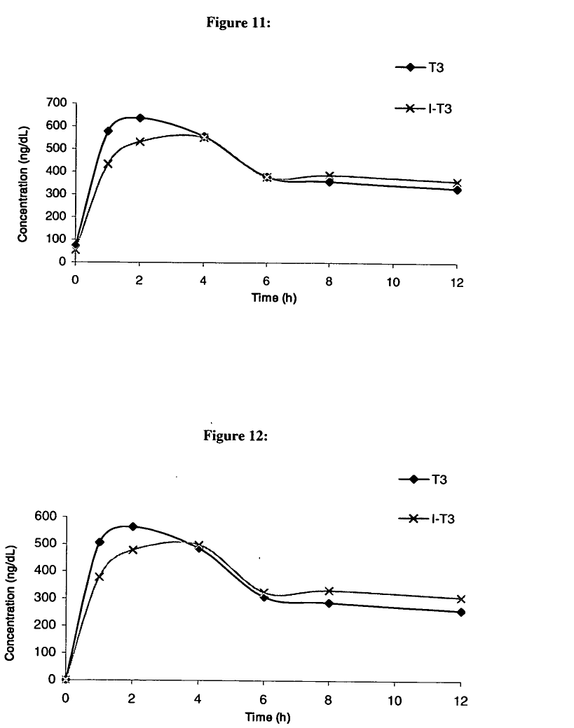

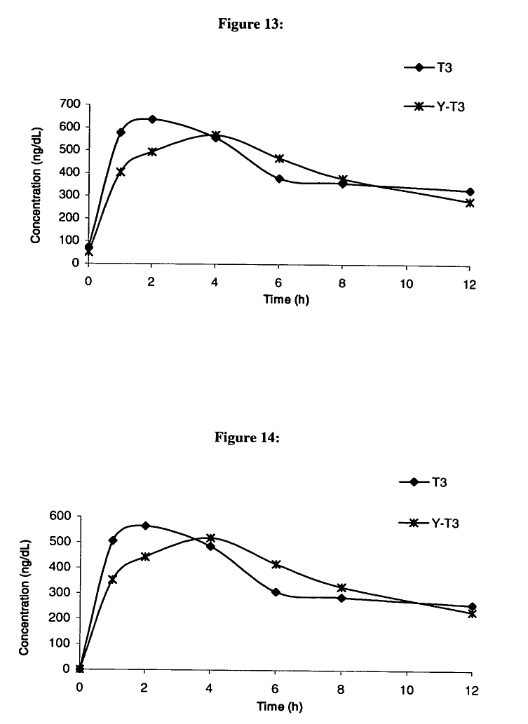

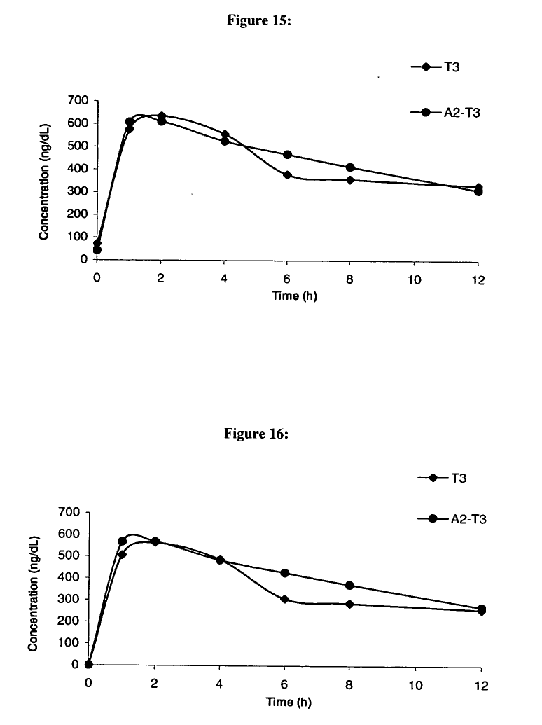

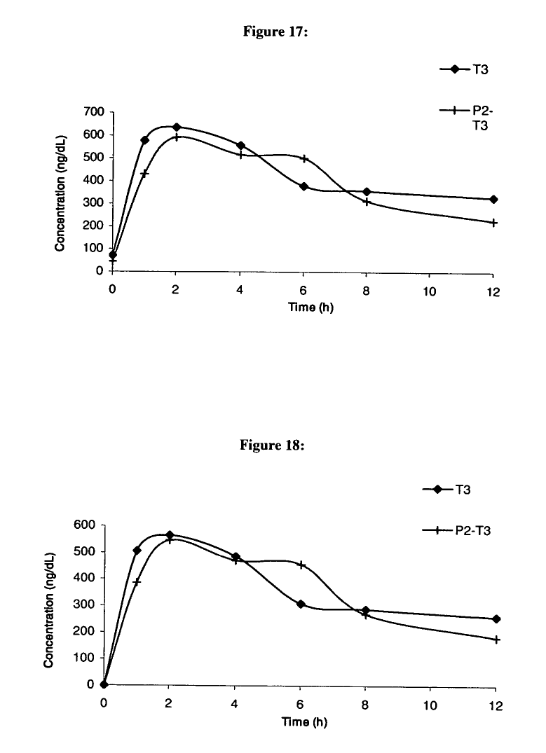

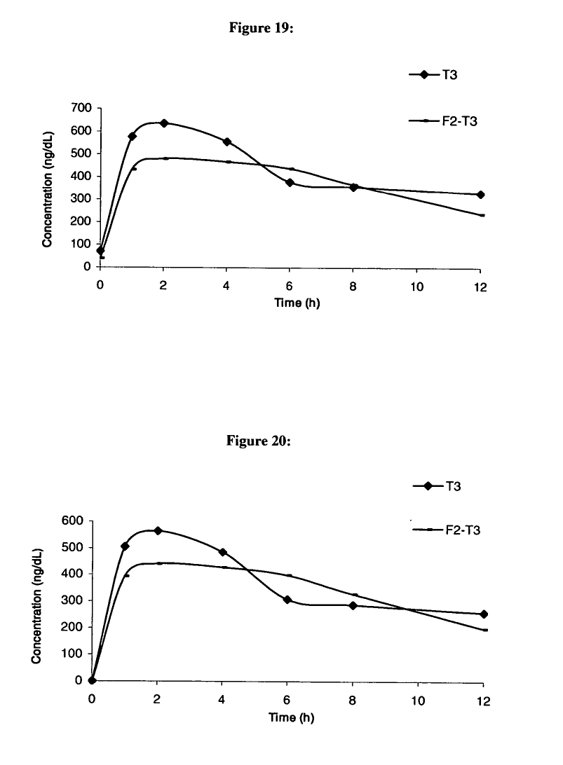

T3 sodium, G-T3, V-T3, I-T3, Y-T3, A2-T3, P2-T3, or F2-T3 were administered at equimolar doses to Sprague-Dawley rats (12 μg/kg T3; HED˜120 μg T3 sodium; n=6, per group) that were hypothyroid due to removal of the thyroid gland approximately one week prior to evaluation. Serum was collected at 0 (pre-dose), 1, 2, 4, 6, 8 and 12 hours post-dose and analyzed for total T3 and TSH by chemiluminescent immunoassay (Immulite CIA). The pharmacokinetic parameters for total T3 and total T3Δ (increase above 0 hour baseline) are summarized in Table 1. A more gradual increase in total T3 (FIGS. 4, 9, 11, 13, 17, and 19) and total T3Δ (FIGS. 5, 10, 12, 14, 18, and 20) accompanied by a sustained release pharmacokinetic profile was observed in rats dosed with G-T3, V-T3, I-T3, Y-T3, P2-T3, or F2-T3 as compared to T3 sodium dosed animals. At 1 hour post dose the mean level of Total T3 was 327.3, 429.5, 432.7, 403.3, 431, and 433.1 ng/ml for G-T3, V-T3, I-T3, Y-T3, P2-T3, and F2-T3 dosed animals respectively as compared to 577 ng/ml for T3 sodium dosed animals. Applicants note that some variation in these values may be present as with A2-T3 (FIGS. 15 and 16). Cₘₐₓ of total T3 for G-T3 was 539 ng/dL compared to 685.5 ng/dL for T3 sodium. The total T3 bioavailability was approximately equivalent for each compound with an AUCₗₐₛₜ value of 4555 ng·h/dL for G-T3 dosed animals compared to 5230 ng·h/dL for T3 sodium dosed animals. Total T3Δ parameters showed profiles similar to total T3 parameters. Total T3 Tₘₐₓ for G-T3 was increased to 4 hours compared to 3.2 hours for T3 sodium.

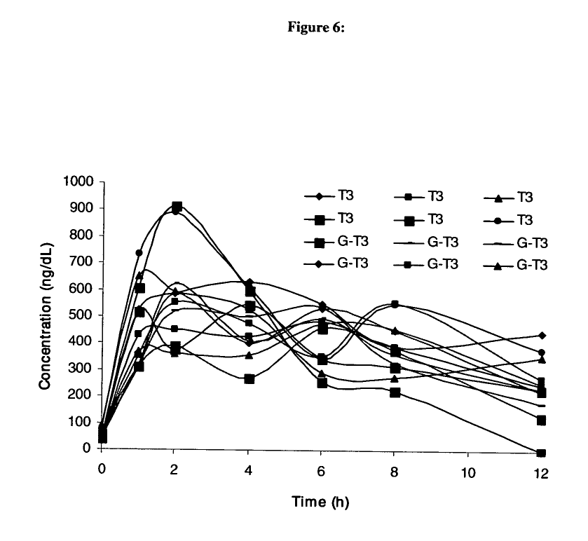

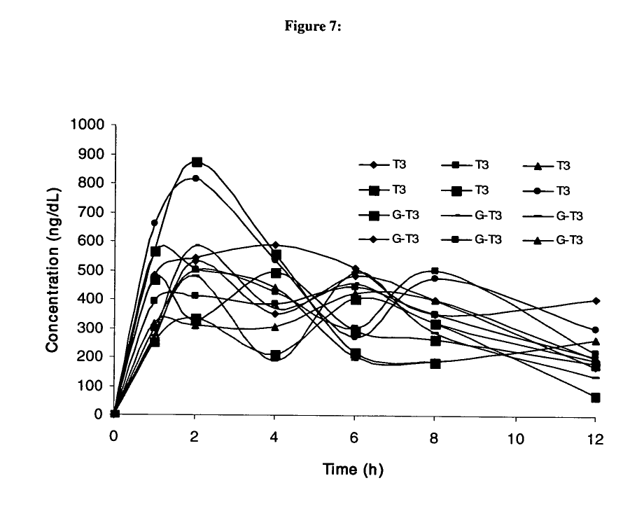

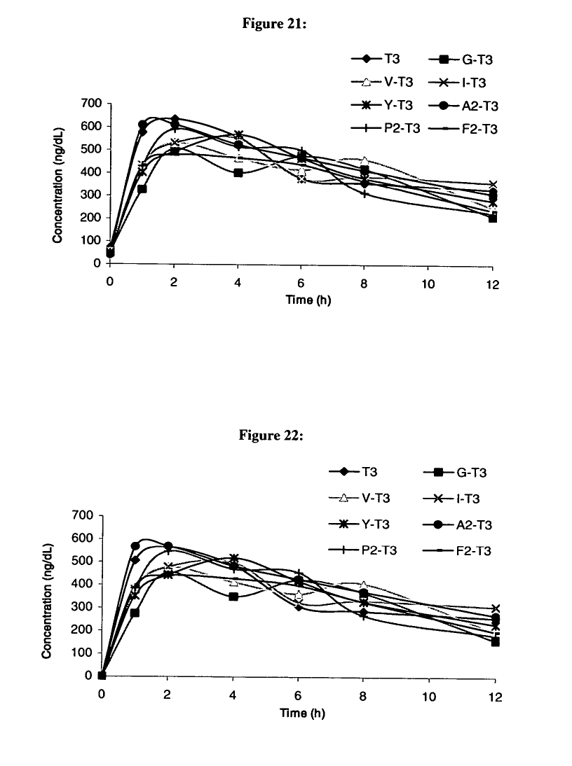

Decreased variability (CV %) in total T3 and total T3Δ AUCₗₐₛₜ and Cₘₐₓ was observed for G-T3 as compared to T3 sodium (Table 1). Notably, variability in total T3Δ Cₘₐₓ for G-T3 (14%) was approximately half the variability observed following administration of T3 sodium (27.9%). Plotting of the individual animal concentration curves for total T3 (FIG. 6) and total T3Δ (FIG. 7) illustrates the decreased variability of T3 absorption from G-T3 compared with T3 sodium. Plotting of the individual conjugate concentration curves for total T3 (FIG. 21) and total T3Δ (FIG. 22) illustrates the decreased variability of T3 absorption from G-T3, V-T3, I-T3, Y-T3, P2-T3, and F2-T3 compared with T3 sodium.

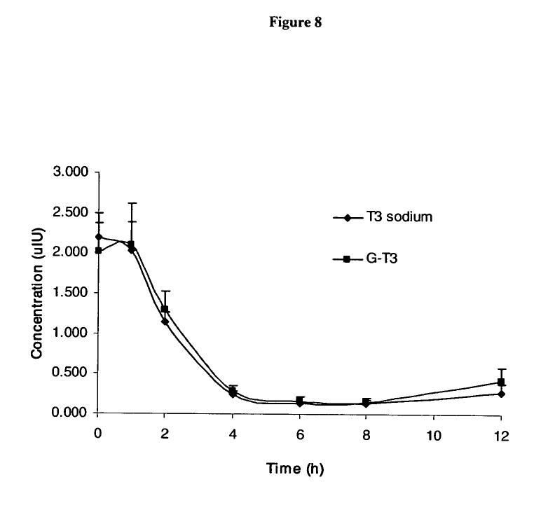

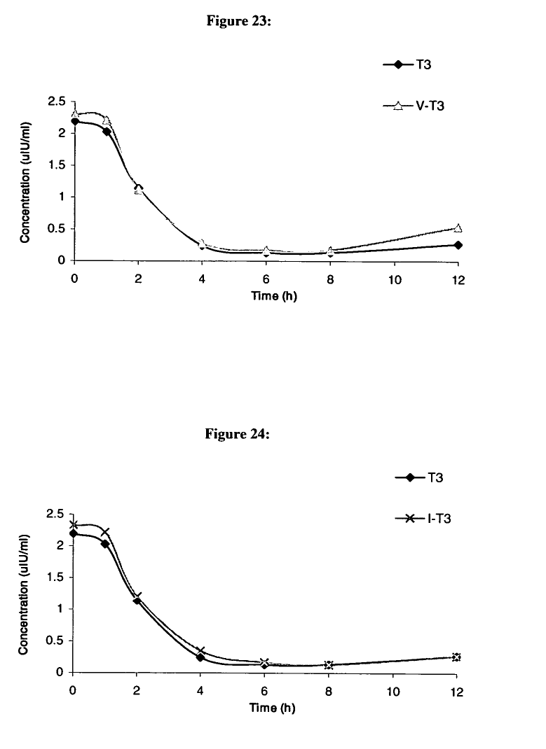

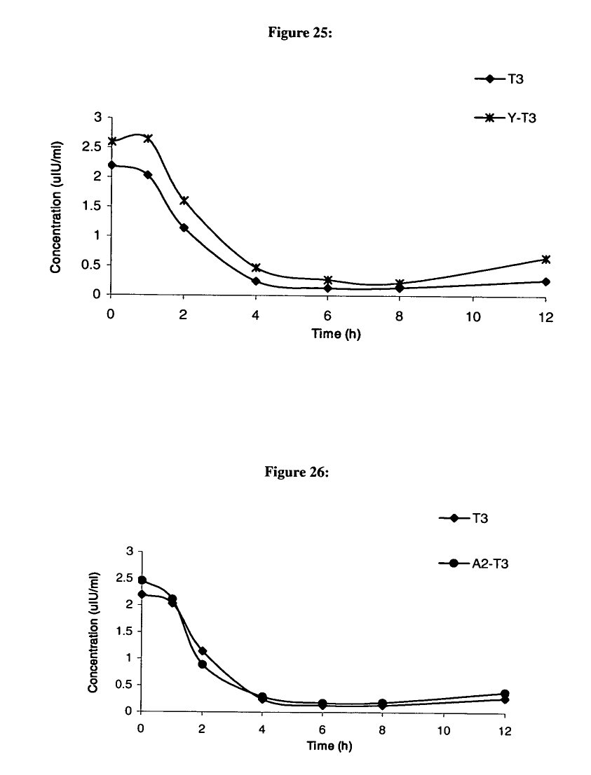

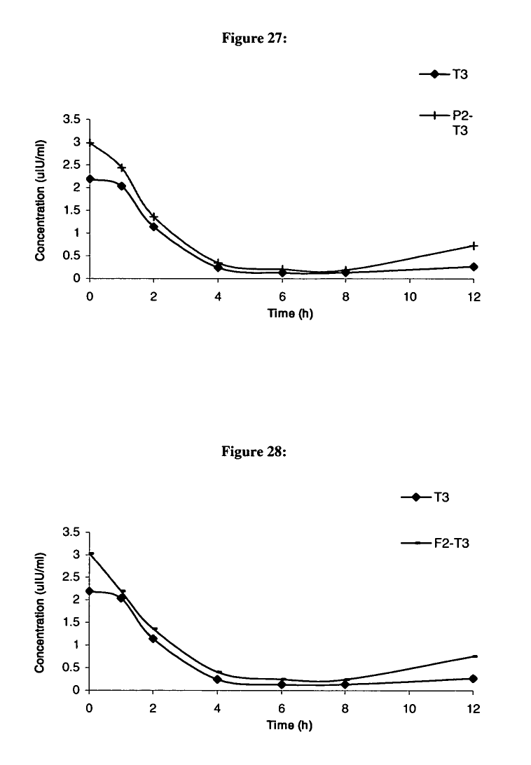

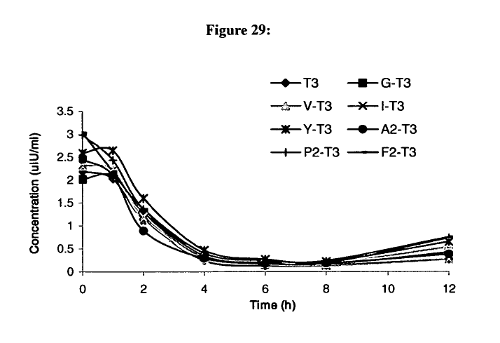

TSH levels decreased rapidly in response to administration of G-T3, V-T3, I-T3, Y-T3, P2-T3, A2T3, F2-T3 or T3 sodium and showed similar pharmacokinetic profiles (FIGS. 8 and 23-29). Levels decreased rapidly after 1 hour post-dose and continued to decline until 6 hours. A small increase in TSH occurred from the 8 hour level to the 12 hour level for each compound.

When compared with T3 sodium, G-T3 afforded delayed release of T3 accompanied by a decrease in total T3 Cₘₐₓ, an increase in Tₘₐₓ, and approximately equal bioavailability. Variability in total T3 and total T3Δ Cₘₐₓ and AUCₗₐₛₜ was decreased by G-T3. Similar decreases in TSH levels were observed in response to administration of G-T3 or T3 sodium.

Prodrugs of the invention may be utilized as a hormone replacement therapy for hypothyroidism. For instance, the prodrug, e.g., Gly-T3, will preferably provide a comparable bioavailability to T3 sodium, but with a slower rate of absorption and a decreased T3 peak level relative to immediate release T3 sodium. Preclinical studies have demonstrated that the prodrugs of the invention, i.e., have delayed absorption, reduced Cₘₐₓ and approximately equal bioavailability when compared to T3 sodium in rats.

TABLE 1
Total T3 and Total T3Δ Pharmacokinetic Parameters

| Parameter | T3 sodium | G-T3 |
|---|---|---|
| Cₘₐₓ (ng/dL) | 685.5 +/− 178.6 | 539 +/− 63.9 |
| CV % | 26.1 | 11.9 |
| % T3 | 100 | 79 |
| Low − High | 484 − 915 | 459 − 625 |
| Range | 431 | 166 |
| Cₘₐₓ Δ (ng/dL) | 629.8 +/− 178.6 | 490.4 +/− 68.6 |
| CV % | 27.9 | 14 |
| % T3 | 100 | 78 |
| Low − High | 444 − 875 | 403 − 534 |
| Range | 431 | 131 |
| AUCₗₐₛₜ (ng · h/dL) | 5230 +/− 821 | 4555 +/− 450 |
| CV % | 16 | 9.9 |
| % T3 | 100 | 89 |
| Low − High | 4292 − 6421 | 3766 − 5072 |
| Range | 2129 | 1306 |
| AUCₗₐₛₜ Δ (ng · h/dL) | 4461 +/− 808 | 3971 +/− 481 |
| CV % | 18.1 | 12.1 |
| % T3 | 100 | 89 |
| Low − High | 3688 − 5526 | 3088 − 4454 |
| Range | 1838 | 1366 |
| Tₘₐₓ | 3.2 +/− 1.8 (1-6) | 4.0 +/− 2.2 (2-6) |
| CV % | 56 | 55 |
| % T3 | 100 | 125 |
| Low − High | 1 − 6 | 2 − 6 |
| Range | 5 | 4 |

TABLE 2
Individual Animal Total T3 (ng/dL)
Following Oral Administration of T3 Sodium

| Time (h) | 1 | 2 | 3 | 4 | 5 | 6 | Mean | SD | CV % |
|---|---|---|---|---|---|---|---|---|---|
| 0 | 0.0 | 0.0 | 89.4 | 0.0 | 50.3 | 74.6 | 35.7 | 41.1 | 115.0 |
| 1 | 523.0 | 432.0 | 650.0 | 605.0 | 517.0 | 735.0 | 577.0 | 108.3 | 18.8 |
| 2 | 585.0 | 452.0 | 595.0 | 915.0 | 377.5 | 890.0 | 635.8 | 222.4 | 35.0 |
| 4 | 630.0 | 426.5 | 530.0 | 600.0 | 544.0 | 610.0 | 556.8 | 74.7 | 13.4 |
| 6 | 550.0 | 484.0 | 292.0 | 259.0 | 343.0 | 346.0 | 379.0 | 113.7 | 30.0 |
| 8 | 391.0 | 390.0 | 276.5 | 225.0 | 316.0 | 550.0 | 358.1 | 114.1 | 31.9 |
| 12 | 442.0 | 245.0 | 353.0 | NS | 231.0 | 376.0 | 329.4 | 89.7 | 27.2 |
| AUCₗₐₛₜ | 5817.5 | 4591.0 | 4766.7 | 3920.5 | 4292.4 | 6421.3 | 4968.2 | 956.0 | 19.2 |
| (ng · h/dL) | | | | | | | | | |
| Cₘₐₓ | 630 | 484 | 650 | 915 | 544 | 890 | 685.5 | 178.6 | 26.1 |
| (ng/dL) | | | | | | | | | |
| Tₘₐₓ (h) | 4 | 6 | 1 | 2 | 4 | 2 | 3.2 | 1.8 | 56.3 |

TABLE 3
Individual Animal Total T3 (ng/dL)
Following Oral Administration of G-T3

| Time (h) | 1 | 2 | 3 | 4 | 5 | 6 | Mean | SD | CV % |
|---|---|---|---|---|---|---|---|---|---|
| 0 | 56.5 | 0.0 | 0.0 | 51.1 | 51.5 | 52.7 | 35.3 | 27.4 | 77.6 |
| 1 | 309.5 | 314.0 | 310.0 | 308.5 | 351.5 | 370.0 | 327.3 | 26.7 | 8.1 |
| 2 | 390.5 | 625.0 | 520.0 | 585.0 | 555.0 | 361.5 | 506.2 | 107.0 | 21.1 |
| 4 | 269.5 | 410.5 | 499.0 | 401.5 | 476.5 | 358.0 | 402.5 | 83.1 | 20.6 |
| 6 | 459.0 | 497.0 | 536.0 | 533.0 | 354.0 | 474.0 | 475.5 | 67.0 | 14.1 |
| 8 | 377.0 | 360.0 | 328.0 | 452.0 | 553.0 | 454.0 | 420.7 | 82.2 | 19.5 |
| 12 | 127.0 | 231.0 | 175.0 | 222.0 | 271.0 | 252.0 | 213.0 | 53.2 | 25.0 |
| AUCₗₐₛₜ | 3765.5 | 4608.5 | 4494.0 | 4880.6 | 5071.8 | 4468.6 | 4548.2 | 449.5 | 9.9 |
| (ng · h/dL) | | | | | | | | | |
| Cₘₐₓ | 459 | 625 | 536 | 585 | 555 | 474 | 539.0 | 63.9 | 11.9 |
| (ng/dL) | | | | | | | | | |
| Tₘₐₓ (h) | 6 | 2 | 6 | 2 | 2 | 6 | 4.0 | 2.2 | 54.8 |

TABLE 4
Individual Animal Total T3Δ (ng/dL)
Following Oral Administration of T3 Sodium

| Time (h) | 1 | 2 | 3 | 4 | 5 | 6 | Mean | SD | CV % |
|---|---|---|---|---|---|---|---|---|---|
| 0 | 0.0 | 0.0 | 0.0 | 0.0 | 0.0 | 0.0 | 0.0 | 0.0 | 0.0 |
| 1 | 483.0 | 392.0 | 560.6 | 565.0 | 466.7 | 660.4 | 521.3 | 93.8 | 18.0 |
| 2 | 545.0 | 412.0 | 505.6 | 875.0 | 327.2 | 815.4 | 580.0 | 219.7 | 37.9 |
| 4 | 590.0 | 386.5 | 440.6 | 560.0 | 493.7 | 535.4 | 501.0 | 76.7 | 15.3 |
| 6 | 510.0 | 444.0 | 202.6 | 219.0 | 292.7 | 271.4 | 323.3 | 125.3 | 38.8 |
| 8 | 351.0 | 350.0 | 187.1 | 185.0 | 265.7 | 475.4 | 302.4 | 112.2 | 37.1 |
| 12 | 402.0 | 205.0 | 263.6 | | 180.7 | 301.4 | 270.5 | 87.5 | 32.3 |
| ΔAUCₗₐₛₜ | 5367.5 | 4131 | 3693.9 | 4360.5 | 3688.8 | 5526.1 | 4461.3 | 807.5 | 18.1 |
| (ng · h/dL) | | | | | | | | | |
| ΔCₘₐₓ | 590 | 444 | 560.6 | 875 | 493.7 | 815.4 | 629.8 | 175.5 | 27.9 |
| (ng/dL) | | | | | | | | | |
| Tₘₐₓ (h) | 4 | 6 | 1 | 2 | 4 | 2 | 3.2 | 1.8 | 56.3 |

TABLE 5
Individual Animal Total T3Δ (ng/dL)
Following Oral Administration of G-T3

| Time (h) | 1 | 2 | 3 | 4 | 5 | 6 | Mean | SD | CV % |
|---|---|---|---|---|---|---|---|---|---|
| 0 | 0.0 | 0.0 | 0.0 | 0.0 | 0.0 | 0.0 | 0.0 | 0.0 | 0.0 |
| 1 | 253.0 | 274.0 | 270.0 | 257.4 | 300.0 | 317.3 | 278.6 | 25.1 | 9.0 |
| 2 | 334.0 | 585.0 | 480.0 | 533.9 | 503.5 | 308.8 | 457.5 | 111.4 | 24.3 |
| 4 | 213.0 | 370.5 | 189.0 | 350.4 | 425.0 | 305.3 | 308.9 | 92.3 | 29.9 |
| 6 | 402.5 | 457.0 | 496.0 | 481.9 | 302.5 | 421.3 | 426.9 | 70.4 | 16.5 |
| 8 | 320.5 | 320.0 | 288.0 | 400.9 | 501.5 | 401.3 | 372.0 | 78.6 | 21.1 |
| 12 | 70.5 | 191.0 | 135.0 | 170.9 | 219.5 | 199.3 | 164.4 | 54.2 | 33.0 |
| ΔAUCₗₐₛₜ | 3087.5 | 4148.5 | 4034 | 4267.4 | 4453.8 | 3836.2 | 3971.2 | 480.7 | 12.1 |
| (ng · h/dL) | | | | | | | | | |
| ΔCₘₐₓ | 402.5 | 585 | 496 | 533.9 | 503.5 | 421.3 | 490.4 | 68.6 | 14.0 |
| (ng/dL) | | | | | | | | | |
| Tₘₐₓ (h) | 6 | 2 | 6 | 2 | 2 | 6 | 4.0 | 2.2 | 55.0 |

TABLE 6
Individual Animal TSH (μIU)
Following Oral Administration of T3 Sodium

| Time | | | | | | | | | |
|---|---|---|---|---|---|---|---|---|---|
| (h) | 1 | 2 | 3 | 4 | 5 | 6 | Mean | SD | CV % |
| 0 | 1.840 | 2.296 | 2.088 | 2.480 | 1.884 | 2.572 | 2.193 | 0.306 | 14.0 |
| 1 | 1.680 | 1.796 | 1.924 | 2.580 | 1.868 | 2.344 | 2.032 | 0.351 | 17.3 |
| 2 | 1.264 | 1.284 | 0.920 | 1.116 | 1.080 | 1.180 | 1.141 | 0.134 | 11.7 |
| 4 | 0.240 | 0.252 | 0.252 | 0.204 | 0.200 | 0.312 | 0.243 | 0.041 | 16.9 |
| 6 | 0.116 | 0.140 | 0.136 | 0.120 | 0.120 | 0.160 | 0.132 | 0.017 | 12.9 |
| 8 | 0.080 | 0.156 | 0.152 | 0.132 | 0.108 | 0.188 | 0.136 | 0.038 | 27.9 |
| 12 | 0.120 | 0.260 | 0.256 | NS | 0.416 | 0.300 | 0.270 | 0.106 | 39.3 |

TABLE 7
Individual Animal TSH (μIU)
Following Oral Administration of G-T3

| Time | | | | | | | | | |
|---|---|---|---|---|---|---|---|---|---|
| (h) | 1 | 2 | 3 | 4 | 5 | 6 | Mean | SD | CV % |
| 0 | 1.680 | 2.584 | 2.304 | 1.956 | 1.860 | 1.740 | 2.021 | 0.353 | 17.5 |
| 1 | 2.660 | 2.564 | 2.392 | 1.960 | 1.612 | 1.400 | 2.098 | 0.522 | 24.9 |
| 2 | 1.340 | 1.472 | 1.648 | 1.160 | 1.104 | 1.008 | 1.289 | 0.243 | 18.9 |
| 4 | 0.364 | 0.352 | 0.296 | 0.216 | 0.276 | 0.248 | 0.292 | 0.058 | 19.9 |
| 6 | 0.232 | 0.172 | 0.152 | 0.112 | 0.205 | 0.100 | 0.162 | 0.052 | 32.1 |
| 8 | 0.232 | 0.172 | 0.136 | 0.124 | 0.168 | 0.076 | 0.151 | 0.053 | 35.1 |
| 12 | 0.540 | 0.612 | 0.224 | 0.548 | 0.268 | 0.372 | 0.427 | 0.162 | 37.9 |

TABLE 8
T3 vs. T3 Conjugates - Total T3 (ng/dL) vs. Time

| Time (h) | T3 | G-T3 | V-T3 | I-T3 | Y-T3 | A2-T3 | P2-T3 | F2-T3 |
|---|---|---|---|---|---|---|---|---|
| 0 | 71.4 | 53 | 54.1 | 54 | 50.7 | 42.2 | 45.7 | 40 |
| 1 | 577 | 327.3 | 429.5 | 432.7 | 403.3 | 609.3 | 431 | 433.1 |
| 2 | 635.8 | 506.2 | 532.8 | 531.8 | 493.5 | 611.3 | 592.1 | 480.8 |
| 4 | 556.8 | 402.5 | 466.5 | 551.9 | 568.8 | 525.1 | 515.5 | 467.7 |
| 6 | 379 | 475.5 | 415.6 | 378.3 | 467.8 | 468.6 | 501 | 438.2 |
| 8 | 358.1 | 420.7 | 461.5 | 385.6 | 378.5 | 414 | 314.3 | 367.5 |
| 12 | 329.4 | 213 | 250.3 | 361 | 280.8 | 310.8 | 226.6 | 238.2 |
| AUC | 5171 | 4557 | 4905 | 4997 | 4939 | 5398 | 4771 | 4565 |
| % | 100 | 88.1 | 94.9 | 96.6 | 95.5 | 104.4 | 92.3 | 88.3 |

TABLE 9
T3 vs. T3 Conjugates - Total T3Δ (ng/dL) vs. Time

| Time (h) | T3 | G-T3 | V-T3 | I-T3 | Y-T3 | A2-T3 | P2-T3 | F2-T3 |
|---|---|---|---|---|---|---|---|---|
| 0 | 0 | 0 | 0 | 0 | 0 | 0 | 0 | 0 |
| 1 | 505.6 | 274.3 | 375.4 | 378.7 | 352.6 | 567.1 | 385.3 | 393.1 |
| 2 | 564.4 | 453.2 | 478.7 | 477.8 | 442.8 | 569.1 | 546.4 | 440.8 |
| 4 | 485.4 | 349.5 | 412.4 | 497.9 | 518.1 | 482.9 | 469.8 | 427.7 |
| 6 | 307.6 | 422.5 | 361.5 | 324.3 | 417.1 | 426.4 | 455.3 | 398.2 |
| 8 | 286.7 | 367.7 | 407.4 | 331.6 | 327.8 | 371.8 | 268.6 | 327.5 |
| 12 | 258 | 160 | 196.2 | 307 | 230.1 | 268.6 | 180.9 | 198.2 |
| dAUC | 4314.3 | 3,921.20 | 4255.9 | 4348.6 | 4330.8 | 4892 | 4222.7 | 4085 |
| % | 100 | 90.9 | 98.6 | 100.8 | 100.4 | 113.4 | 97.9 | 94.7 |

TABLE 10
T3 vs. T3 Conjugates - TSH (μIU) vs. Time

| Time | | | | | | | | |
|---|---|---|---|---|---|---|---|---|
| (h) | T3 | G-T3 | V-T3 | I-T3 | Y-T3 | A2-T3 | P2-T3 | F2-T3 |
| 0 | 2.193 | 2.021 | 2.309 | 2.331 | 2.595 | 2.459 | 2.988 | 3.035 |
| 1 | 2.032 | 2.098 | 2.212 | 2.218 | 2.648 | 2.11 | 2.438 | 2.192 |
| 2 | 1.141 | 1.289 | 1.123 | 1.203 | 1.601 | 0.889 | 1.363 | 1.364 |
| 4 | 0.243 | 0.292 | 0.282 | 0.351 | 0.471 | 0.292 | 0.346 | 0.405 |
| 6 | 0.132 | 0.162 | 0.174 | 0.169 | 0.27 | 0.174 | 0.21 | 0.246 |
| 8 | 0.136 | 0.151 | 0.175 | 0.131 | 0.221 | 0.182 | 0.189 | 0.242 |
| 12 | 0.27 | 0.427 | 0.54 | 0.271 | 0.651 | 0.381 | 0.735 | 0.757 |

1. A composition comprising at least one peptidic-iodothyronine or salt thereof and at least one non-peptidic iodothyronine or salt thereof.

2. The composition of claim 1 comprising Gly-T3 or salt thereof and T4 or salt thereof.

3. The composition of claim 1 comprising Gly-T4 or salt thereof and T3 or salt thereof.

4. A composition comprising Gly-T3 or salt thereof and Gly-T4 or salt thereof.

5. A method of treating a thyroid disorder comprising orally administering to a human patient an iodothyronine prodrug or salt thereof wherein said prodrug comprises a single iodothyronine covalently bound through to the C-terminus of peptide carrier wherein the carrier peptide is fewer than 5 amino acid.

6. The method of claim 5, wherein said iodothyronine is T3.

7. The method of claim 5, wherein said iodothyronine is T4.

8. The method of claim 6, wherein said carrier peptide is a single amino acid.

9. The method of claim 6, wherein said carrier peptide is Gly, Lys, Glu, Ile, Tyr, Val, Ala-Ala, Pro-Pro, Glu-Glu or Phe-Phe.

10. The method of claim 8, wherein said prodrug is in salt form.

11. The method of claim 8, wherein said salt form is a HCl, acetate, sulfate, mesylate, citrate, phosphate, or tartrate salt.

12. The method of claim 8, wherein said salt form is an HCl salt.

13. The method of claim 8, wherein said prodrug is Gly-T3 or a salt thereof.

14. The method of claim 8 or 13, wherein said condition is hypothyroidism.

15. The method of claim 8 or 13, wherein said condition is depression.

16. Gly-T3.

17. Ile-T3.

18. Tyr-T3.

19. Ala-Ala-T3.

20. Val-T3.

21. Pro-Pro-T3.

22. Phe-Phe-T3.

23. Gly-T4.

24. Ile-T4.

25. Tyr-T4.

26. Ala-Ala-T4.

27. Val-T4.

28. Pro-Pro-T4.

29. Phe-Phe-T4.

30. T4-Glu.

31. T4-Glu-Glu.

32. T4-Lys.

33. Glu-T4.

34. Glu-Glu-T4.

35. Lys-T4.


SEQUENCE LISTING (18 sequences)

SEQ ID NO: 1 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
AAA

SEQ ID NO: 2 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
RRR

SEQ ID NO: 3 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
NNN

SEQ ID NO: 4 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
QQQ

SEQ ID NO: 5 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
GGG

SEQ ID NO: 6 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
EEE

SEQ ID NO: 7 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
HHH

SEQ ID NO: 8 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
III

SEQ ID NO: 9 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
LLL

SEQ ID NO: 10 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
KKK

SEQ ID NO: 11 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
MMM

SEQ ID NO: 12 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
FFF

SEQ ID NO: 13 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
PPP

SEQ ID NO: 14 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
SSS

SEQ ID NO: 15 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
TTT

SEQ ID NO: 16 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
WWW

SEQ ID NO: 17 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
YYY

SEQ ID NO: 18 - protein, 3 aa, Artificial Sequence
  Synthetic Carrier
  MISC_FEATURE (1)..(3): T3 or T4 attached through the carboxy group exclusively to the N-terminus of the sequence or T3 or T4 attached through the amino group exclusively to the C-terminus of the sequence
VVV